SEQUENCE LISTING (313 sequences)

SEQ ID NO: 1 - DNA, 23 bp, Artificial Sequence
  Description of Artificial Sequence Synthetic primer
atagttcccctaggagaggtggg

SEQ ID NO: 2 - DNA, 20 bp, Artificial Sequence
  Description of Artificial Sequence Synthetic primer
cagcctgaccccacagtccc

SEQ ID NO: 3 - DNA, 23 bp, Artificial Sequence
  Description of Artificial Sequence Synthetic primer
atagtttttttaggagaggtggg

SEQ ID NO: 4 - DNA, 20 bp, Artificial Sequence
  Description of Artificial Sequence Synthetic primer
caacctaaccccacaatccc

SEQ ID NO: 5 - DNA, 25 bp, Artificial Sequence
  Description of Artificial Sequence Synthetic primer
gctgagggtgactgtgctgtgaggc

SEQ ID NO: 6 - DNA, 25 bp, Artificial Sequence
  Description of Artificial Sequence Synthetic primer
cccaggctccctgcaggttcacccc

SEQ ID NO: 7 - DNA, 25 bp, Artificial Sequence
  Description of Artificial Sequence Synthetic primer
gttgagggtgattgtgttgtgaggt

SEQ ID NO: 8 - DNA, 25 bp, Artificial Sequence
  Description of Artificial Sequence Synthetic primer
cccaaactccctacaaattcacccc

SEQ ID NO: 9 - DNA, 25 bp, Artificial Sequence
  Description of Artificial Sequence Synthetic primer
ggtgagcccctggaaaaggaggggg

SEQ ID NO: 10 - DNA, 25 bp, Artificial Sequence
  Description of Artificial Sequence Synthetic primer
ccctttactgcccatctctgccatc

SEQ ID NO: 11 - DNA, 25 bp, Artificial Sequence
  Description of Artificial Sequence Synthetic primer
ggtgagtttttggaaaaggaggggg

SEQ ID NO: 12 - DNA, 25 bp, Artificial Sequence
  Description of Artificial Sequence Synthetic primer
ccctttactacccatctctaccatc

SEQ ID NO: 13 - DNA, 24 bp, Artificial Sequence
  Description of Artificial Sequence Synthetic primer
tggcctgggactcttaagggtcag

SEQ ID NO: 14 - DNA, 27 bp, Artificial Sequence
  Description of Artificial Sequence Synthetic primer
ttggcagagacaaaagggcaagaagcc

SEQ ID NO: 15 - DNA, 24 bp, Artificial Sequence
  Description of Artificial Sequence Synthetic primer
tggtttgggatttttaagggttag

SEQ ID NO: 16 - DNA, 27 bp, Artificial Sequence
  Description of Artificial Sequence Synthetic primer
ttaacaaaaacaaaaaaacaaaaaacc

SEQ ID NO: 17 - DNA, 25 bp, Artificial Sequence
  Description of Artificial Sequence Synthetic primer
cccctagggtggaacagaaatggct

SEQ ID NO: 18 - DNA, 25 bp, Artificial Sequence
  Description of Artificial Sequence Synthetic primer
atctcaatagctgcatttccagctc

SEQ ID NO: 19 - DNA, 25 bp, Artificial Sequence
  Description of Artificial Sequence Synthetic primer
tttttagggtggaatagaaatggtt

SEQ ID NO: 20 - DNA, 25 bp, Artificial Sequence
  Description of Artificial Sequence Synthetic primer
atctcaataactacatttccaactc

SEQ ID NO: 21 - DNA, 25 bp, Artificial Sequence
  Description of Artificial Sequence Synthetic primer
gagctggaaatgcagctattgagat

SEQ ID NO: 22 - DNA, 25 bp, Artificial Sequence
  Description of Artificial Sequence Synthetic primer
ccacggctcctttgtccccaaatcc

SEQ ID NO: 23 - DNA, 25 bp, Artificial Sequence
  Description of Artificial Sequence Synthetic primer
gagttggaaatgtagttattgagat

SEQ ID NO: 24 - DNA, 25 bp, Artificial Sequence
  Description of Artificial Sequence Synthetic primer
ccacractcctttatccccaaatcc

SEQ ID NO: 25 - DNA, 25 bp, Artificial Sequence
  Description of Artificial Sequence Synthetic primer
ggtctagagagctgggtctagaact

SEQ ID NO: 26 - DNA, 22 bp, Artificial Sequence
  Description of Artificial Sequence Synthetic primer
cccctgcccctcccctgtcccc

SEQ ID NO: 27 - DNA, 25 bp, Artificial Sequence
  Description of Artificial Sequence Synthetic primer
ggtttagagagttgggtttagaatt

SEQ ID NO: 28 - DNA, 22 bp, Artificial Sequence
  Description of Artificial Sequence Synthetic primer
cccctacccctcccctatcccc

SEQ ID NO: 29 - DNA, 25 bp, Artificial Sequence
  Description of Artificial Sequence Synthetic primer
ggtacttccttcaaagctgagggag

SEQ ID NO: 30 - DNA, 22 bp, Artificial Sequence
  Description of Artificial Sequence Synthetic primer
caaccaggctcccctagaccac

SEQ ID NO: 31 - DNA, 25 bp, Artificial Sequence
  Description of Artificial Sequence Synthetic primer
ggtattttttttaaagttgagggag

SEQ ID NO: 32 - DNA, 22 bp, Artificial Sequence
  Description of Artificial Sequence Synthetic primer
caaccaaactcccctaaaccac

SEQ ID NO: 33 - DNA, 24 bp, Artificial Sequence
  Description of Artificial Sequence Synthetic primer
gaggcacaagggcactctggaggg

SEQ ID NO: 34 - DNA, 26 bp, Artificial Sequence
  Description of Artificial Sequence Synthetic primer
cagcacatagaggggaccttcctggc

SEQ ID NO: 35 - DNA, 24 bp, Artificial Sequence
  Description of Artificial Sequence Synthetic primer
gaggtataagggtattttggaggg

SEQ ID NO: 36 - DNA, 26 bp, Artificial Sequence
  Description of Artificial Sequence Synthetic primer
caacacataaaaaaaaccttcctaac

SEQ ID NO: 37 - DNA, 26 bp, Artificial Sequence
  Description of Artificial Sequence Synthetic primer
gtgtaggactccattcagctcattgg

SEQ ID NO: 38 - DNA, 27 bp, Artificial Sequence
  Description of Artificial Sequence Synthetic primer
ccgaagtgacagaataggcccttgtgc

SEQ ID NO: 39 - DNA, 26 bp, Artificial Sequence
  Description of Artificial Sequence Synthetic primer
gtgtaggattttatttagtttattgg

SEQ ID NO: 40 - DNA, 27 bp, Artificial Sequence
  Description of Artificial Sequence Synthetic primer
ccraaataacaaaataaacccttatac

SEQ ID NO: 41 - DNA, 25 bp, Artificial Sequence
  Description of Artificial Sequence Synthetic primer
ggagctgggggtagacaggtgcaag

SEQ ID NO: 42 - DNA, 30 bp, Artificial Sequence
  Description of Artificial Sequence Synthetic primer
caatggctgcactcaatatccgggctggac

SEQ ID NO: 43 - DNA, 25 bp, Artificial Sequence
  Description of Artificial Sequence Synthetic primer
ggagttgggggtagataggtgtaag

SEQ ID NO: 44 - DNA, 30 bp, Artificial Sequence
  Description of Artificial Sequence Synthetic primer
caataactacactcaatatccraactaaac

SEQ ID NO: 45 - DNA, 23 bp, Artificial Sequence
  Description of Artificial Sequence Synthetic primer
gaggaaggtctgtcacagggagg

SEQ ID NO: 46 - DNA, 24 bp, Artificial Sequence
  Description of Artificial Sequence Synthetic primer
cgccattaacaaggccagaacccc

SEQ ID NO: 47 - DNA, 23 bp, Artificial Sequence
  Description of Artificial Sequence Synthetic primer
gaggaaggtttgttatagggagg

SEQ ID NO: 48 - DNA, 24 bp, Artificial Sequence
  Description of Artificial Sequence Synthetic primer
crccattaacaaaaccaaaacccc

SEQ ID NO: 49 - DNA, 25 bp, Artificial Sequence
  Description of Artificial Sequence Synthetic primer
gaaaggcactagctctctacaaagc

SEQ ID NO: 50 - DNA, 25 bp, Artificial Sequence
  Description of Artificial Sequence Synthetic primer
gggcttgttcaaaccagcttggagc

SEQ ID NO: 51 - DNA, 25 bp, Artificial Sequence
  Description of Artificial Sequence Synthetic primer
gaaaggtattagttttttataaagt

SEQ ID NO: 52 - DNA, 25 bp, Artificial Sequence
  Description of Artificial Sequence Synthetic primer
aaacttattcaaaccaacttaaaac

SEQ ID NO: 53 - DNA, 26 bp, Artificial Sequence
  Description of Artificial Sequence Synthetic primer
gaaagcaaagaaagaggtactgttta

SEQ ID NO: 54 - DNA, 25 bp, Artificial Sequence
  Description of Artificial Sequence Synthetic primer
ctggcagcagcacagacactgccac

SEQ ID NO: 55 - DNA, 26 bp, Artificial Sequence
  Description of Artificial Sequence Synthetic primer
gaaagtaaagaaagaggtattgttta

SEQ ID NO: 56 - DNA, 25 bp, Artificial Sequence
  Description of Artificial Sequence Synthetic primer
ctaacaacaacacaaacactaccac

SEQ ID NO: 57 - DNA, 27 bp, Artificial Sequence
  Description of Artificial Sequence Synthetic primer
aggcaagggcacccaagttcccagagt

SEQ ID NO: 58 - DNA, 24 bp, Artificial Sequence
  Description of Artificial Sequence Synthetic primer
ctgactgcagagcctgggtgcccc

SEQ ID NO: 59 - DNA, 27 bp, Artificial Sequence
  Description of Artificial Sequence Synthetic primer
aggtaagggtatttaagtttttagagt

SEQ ID NO: 60 - DNA, 24 bp, Artificial Sequence
  Description of Artificial Sequence Synthetic primer
ctaactacaaaacctaaatacccc

SEQ ID NO: 61 - DNA, 23 bp, Artificial Sequence
  Description of Artificial Sequence Synthetic primer
gcaggcagtgaaggagatggctg

SEQ ID NO: 62 - DNA, 27 bp, Artificial Sequence
  Description of Artificial Sequence Synthetic primer
tacctggggtggaccaagcacaggtca

SEQ ID NO: 63 - DNA, 23 bp, Artificial Sequence
  Description of Artificial Sequence Synthetic primer
gtaggtagtgaaggagatggttg

SEQ ID NO: 64 - DNA, 27 bp, Artificial Sequence
  Description of Artificial Sequence Synthetic primer
tacctaaaataaaccaaacacaaatca

SEQ ID NO: 65 - DNA, 24 bp, Artificial Sequence
  Description of Artificial Sequence Synthetic primer
tgaggagcagagctcagcttgtgc

SEQ ID NO: 66 - DNA, 25 bp, Artificial Sequence
  Description of Artificial Sequence Synthetic primer
catctcattgagggtggcctcctcc

SEQ ID NO: 67 - DNA, 24 bp, Artificial Sequence
  Description of Artificial Sequence Synthetic primer
tgaggagtagagtttagtttgtgt

SEQ ID NO: 68 - DNA, 25 bp, Artificial Sequence
  Description of Artificial Sequence Synthetic primer
catctcattaaaaataacctcctcc

SEQ ID NO: 69 - DNA, 25 bp, Artificial Sequence
  Description of Artificial Sequence Synthetic primer
gctcaaggcacactagggtccaggc

SEQ ID NO: 70 - DNA, 23 bp, Artificial Sequence
  Description of Artificial Sequence Synthetic primer
gcttctttctgcacatcaagcac

SEQ ID NO: 71 - DNA, 25 bp, Artificial Sequence
  Description of Artificial Sequence Synthetic primer
gtttaaggtatattagggtttaggt

SEQ ID NO: 72 - DNA, 23 bp, Artificial Sequence
  Description of Artificial Sequence Synthetic primer
acttctttctacacatcaaacac

SEQ ID NO: 73 - DNA, 19 bp, Artificial Sequence
  Description of Artificial Sequence Synthetic primer
ggagggagagagggaggag

SEQ ID NO: 74 - DNA, 20 bp, Artificial Sequence
  Description of Artificial Sequence Synthetic primer
cccacttccatgaccatggc

SEQ ID NO: 75 - DNA, 19 bp, Artificial Sequence
  Description of Artificial Sequence Synthetic primer
ggagggagagagggaggag

SEQ ID NO: 76 - DNA, 20 bp, Artificial Sequence
  Description of Artificial Sequence Synthetic primer
cccacttccataaccataac

SEQ ID NO: 77 - DNA, 30 bp, Artificial Sequence
  Description of Artificial Sequence Synthetic primer
aagacatgaaaagataataagaaaaaagtg

SEQ ID NO: 78 - DNA, 29 bp, Artificial Sequence
  Description of Artificial Sequence Synthetic primer
cttcctcccctgcaagccttccacagccc

SEQ ID NO: 79 - DNA, 30 bp, Artificial Sequence
  Description of Artificial Sequence Synthetic primer
aagatatgaaaagataataagaaaaaagtg

SEQ ID NO: 80 - DNA, 29 bp, Artificial Sequence
  Description of Artificial Sequence Synthetic primer
cttcctcccctacaaaccttccacaaccc

SEQ ID NO: 81 - DNA, 25 bp, Artificial Sequence
  Description of Artificial Sequence Synthetic primer
tccccaggagaccaaagaggttgga

SEQ ID NO: 82 - DNA, 25 bp, Artificial Sequence
  Description of Artificial Sequence Synthetic primer
tggggctctgggcccagagagggtg

SEQ ID NO: 83 - DNA, 25 bp, Artificial Sequence
  Description of Artificial Sequence Synthetic primer
tttttaggagattaaagaggttgga

SEQ ID NO: 84 - DNA, 25 bp, Artificial Sequence
  Description of Artificial Sequence Synthetic primer
taaaactctaaacccaaaaaaaata

SEQ ID NO: 85 - DNA, 28 bp, Artificial Sequence
  Description of Artificial Sequence Synthetic primer
gcccccttgccctggctgtaatggagac

SEQ ID NO: 86 - DNA, 27 bp, Artificial Sequence
  Description of Artificial Sequence Synthetic primer
ctgggaacttgcacagcccacccagac

SEQ ID NO: 87 - DNA, 28 bp, Artificial Sequence
  Description of Artificial Sequence Synthetic primer
gtttttttgttttggttgtaatggagat

SEQ ID NO: 88 - DNA, 27 bp, Artificial Sequence
  Description of Artificial Sequence Synthetic primer
ctaaaaacttacacaacccacccaaac

SEQ ID NO: 89 - DNA, 25 bp, Artificial Sequence
  Description of Artificial Sequence Synthetic primer
gttaggatctggctggtgtctcaag

SEQ ID NO: 90 - DNA, 26 bp, Artificial Sequence
  Description of Artificial Sequence Synthetic primer
ccagggaagcccccagtttacactgc

SEQ ID NO: 91 - DNA, 25 bp, Artificial Sequence
  Description of Artificial Sequence Synthetic primer
gttaggatttggttggtgttttaag

SEQ ID NO: 92 - DNA, 26 bp, Artificial Sequence
  Description of Artificial Sequence Synthetic primer
ccaaaaaaacccccaatttacactac

SEQ ID NO: 93 - DNA, 25 bp, Artificial Sequence
  Description of Artificial Sequence Synthetic primer
agccagaggctgaggggaggctttg

SEQ ID NO: 94 - DNA, 26 bp, Artificial Sequence
  Description of Artificial Sequence Synthetic primer
tgaggtgtgctcacagcctcagcttc

SEQ ID NO: 95 - DNA, 25 bp, Artificial Sequence
  Description of Artificial Sequence Synthetic primer
agttagaggttgaggggaggttttg

SEQ ID NO: 96 - DNA, 26 bp, Artificial Sequence
  Description of Artificial Sequence Synthetic primer
taaaatatactcacaacctcaacttc

SEQ ID NO: 97 - DNA, 27 bp, Artificial Sequence
  Description of Artificial Sequence Synthetic primer
ttaaaataacacttgaaataggtgtca

SEQ ID NO: 98 - DNA, 24 bp, Artificial Sequence
  Description of Artificial Sequence Synthetic primer
ccttcatccccacaagtgccctcc

SEQ ID NO: 99 - DNA, 27 bp, Artificial Sequence
  Description of Artificial Sequence Synthetic primer
ttaaaataatatttgaaataggtgtta

SEQ ID NO: 100 - DNA, 24 bp, Artificial Sequence
  Description of Artificial Sequence Synthetic primer
ccttcatccccacaaataccctcc

SEQ ID NO: 101 - DNA, 26 bp, Artificial Sequence
  Description of Artificial Sequence Synthetic primer
gaggggcatccctcagtggagggagc

SEQ ID NO: 102 - DNA, 26 bp, Artificial Sequence
  Description of Artificial Sequence Synthetic primer
ctacctaagcatgcatgacctgaccc

SEQ ID NO: 103 - DNA, 26 bp, Artificial Sequence
  Description of Artificial Sequence Synthetic primer
gaggggtattttttagtggagggagt

SEQ ID NO: 104 - DNA, 26 bp, Artificial Sequence
  Description of Artificial Sequence Synthetic primer
ctacctaaacatacataacctaaccc

SEQ ID NO: 105 - DNA, 30 bp, Artificial Sequence
  Description of Artificial Sequence Synthetic primer
agtctggaggtcagacgaacagcaaattgg

SEQ ID NO: 106 - DNA, 28 bp, Artificial Sequence
  Description of Artificial Sequence Synthetic primer
catccttgcctgggtgttcagccctgtc

SEQ ID NO: 107 - DNA, 30 bp, Artificial Sequence
  Description of Artificial Sequence Synthetic primer
agtttggaggttagaygaatagtaaattgg

SEQ ID NO: 108 - DNA, 28 bp, Artificial Sequence
  Description of Artificial Sequence Synthetic primer
catccttacctaaatattcaaccctatc

SEQ ID NO: 109 - DNA, 23 bp, Artificial Sequence
  Description of Artificial Sequence Synthetic primer
gaaaagatggccccaggagccag

SEQ ID NO: 110 - DNA, 20 bp, Artificial Sequence
  Description of Artificial Sequence Synthetic primer
ccccccacttcctggtgccc

SEQ ID NO: 111 - DNA, 23 bp, Artificial Sequence
  Description of Artificial Sequence Synthetic primer
gaaaagatggttttaggagttag

SEQ ID NO: 112 - DNA, 20 bp, Artificial Sequence
  Description of Artificial Sequence Synthetic primer
ccccccacttcctaataccc

SEQ ID NO: 113 - DNA, 26 bp, Artificial Sequence
  Description of Artificial Sequence Synthetic primer
tgtcccctgggctgtagtggagtggc

SEQ ID NO: 114 - DNA, 29 bp, Artificial Sequence
  Description of Artificial Sequence Synthetic primer
caggtgtggactgagggcttgtagccacc

SEQ ID NO: 115 - DNA, 26 bp, Artificial Sequence
  Description of Artificial Sequence Synthetic primer
tgttttttgggttgtagtggagtggt

SEQ ID NO: 116 - DNA, 29 bp, Artificial Sequence
  Description of Artificial Sequence Synthetic primer
caaatataaactaaaaacttataaccacc

SEQ ID NO: 117 - DNA, 26 bp, Artificial Sequence
  Description of Artificial Sequence Synthetic primer
tgaaacccctcactcatgtgcctggg

SEQ ID NO: 118 - DNA, 26 bp, Artificial Sequence
  Description of Artificial Sequence Synthetic primer
agggctgttccattttagtgcacatc

SEQ ID NO: 119 - DNA, 26 bp, Artificial Sequence
  Description of Artificial Sequence Synthetic primer
tgaaattttttatttatgtgtttggg

SEQ ID NO: 120 - DNA, 26 bp, Artificial Sequence
  Description of Artificial Sequence Synthetic primer
aaaactattccattttaatacacatc

SEQ ID NO: 121 - DNA, 22 bp, Artificial Sequence
  Description of Artificial Sequence Synthetic primer
ggagggagaggggatccagctc

SEQ ID NO: 122 - DNA, 24 bp, Artificial Sequence
  Description of Artificial Sequence Synthetic primer
ccaggacccctcccctgcccactc

SEQ ID NO: 123 - DNA, 22 bp, Artificial Sequence
  Description of Artificial Sequence Synthetic primer
ggagggagaggggatttagttt

SEQ ID NO: 124 - DNA, 24 bp, Artificial Sequence
  Description of Artificial Sequence Synthetic primer
ccaaaacccctcccctacccactc

SEQ ID NO: 125 - DNA, 26 bp, Artificial Sequence
  Description of Artificial Sequence Synthetic primer
gatgggtaagagagtgaaccctgtgg

SEQ ID NO: 126 - DNA, 27 bp, Artificial Sequence
  Description of Artificial Sequence Synthetic primer
ctcacccccagaagcagcaacagcagc

SEQ ID NO: 127 - DNA, 26 bp, Artificial Sequence
  Description of Artificial Sequence Synthetic primer
gatgggtaagagagtgaattttgtgg

SEQ ID NO: 128 - DNA, 27 bp, Artificial Sequence
  Description of Artificial Sequence Synthetic primer
ctcacccccaaaaacaacaacaacaac

SEQ ID NO: 129 - DNA, 25 bp, Artificial Sequence
  Description of Artificial Sequence Synthetic primer
ttgccacactctgaccaatcaggag

SEQ ID NO: 130 - DNA, 24 bp, Artificial Sequence
  Description of Artificial Sequence Synthetic primer
accagagaggagctctggggtctc

SEQ ID NO: 131 - DNA, 25 bp, Artificial Sequence
  Description of Artificial Sequence Synthetic primer
ttgttatattttgattaattaggag

SEQ ID NO: 132 - DNA, 24 bp, Artificial Sequence
  Description of Artificial Sequence Synthetic primer
accaaaaaaaaactctaaaatctc

SEQ ID NO: 133 - DNA, 24 bp, Artificial Sequence
  Description of Artificial Sequence Synthetic primer
gggaccggctggcctgctggaact

SEQ ID NO: 134 - DNA, 24 bp, Artificial Sequence
  Description of Artificial Sequence Synthetic primer
cttcctgggcccctccagggacac

SEQ ID NO: 135 - DNA, 24 bp, Artificial Sequence
  Description of Artificial Sequence Synthetic primer
gggattggttggtttgttggaatt

SEQ ID NO: 136 - DNA, 24 bp, Artificial Sequence
  Description of Artificial Sequence Synthetic primer
cttcctaaacccctccaaaaacac

SEQ ID NO: 137 - DNA, 25 bp, Artificial Sequence
  Description of Artificial Sequence Synthetic primer
ggctgggcgttccacaggccaagtg

SEQ ID NO: 138 - DNA, 23 bp, Artificial Sequence
  Description of Artificial Sequence Synthetic primer
ccgggacactaggcagctgctcc

SEQ ID NO: 139 - DNA, 25 bp, Artificial Sequence
  Description of Artificial Sequence Synthetic primer
ggttgggygttttataggttaagtg

SEQ ID NO: 140 - DNA, 23 bp, Artificial Sequence
  Description of Artificial Sequence Synthetic primer
ccraaacactaaacaactactcc

SEQ ID NO: 141 - DNA, 25 bp, Artificial Sequence
  Description of Artificial Sequence Synthetic primer
ttcacccctggtgaaactaggggag

SEQ ID NO: 142 - DNA, 23 bp, Artificial Sequence
  Description of Artificial Sequence Synthetic primer
cagaggaagtccccacccctctc

SEQ ID NO: 143 - DNA, 25 bp, Artificial Sequence
  Description of Artificial Sequence Synthetic primer
tttatttttggtgaaattaggggag

SEQ ID NO: 144 - DNA, 23 bp, Artificial Sequence
  Description of Artificial Sequence Synthetic primer
caaaaaaaatccccacccctctc

SEQ ID NO: 145 - DNA, 26 bp, Artificial Sequence
  Description of Artificial Sequence Synthetic primer
cataaaagtctgagtgagtccctggc

SEQ ID NO: 146 - DNA, 24 bp, Artificial Sequence
  Description of Artificial Sequence Synthetic primer
gccatgttccccccatttccaggg

SEQ ID NO: 147 - DNA, 26 bp, Artificial Sequence
  Description of Artificial Sequence Synthetic primer
tataaaagtttgagtgagtttttggt

SEQ ID NO: 148 - DNA, 24 bp, Artificial Sequence
  Description of Artificial Sequence Synthetic primer
accatattccccccatttccaaaa

SEQ ID NO: 149 - DNA, 25 bp, Artificial Sequence
  Description of Artificial Sequence Synthetic primer
agaaaagtttgtacagagggtggaa

SEQ ID NO: 150 - DNA, 25 bp, Artificial Sequence
  Description of Artificial Sequence Synthetic primer
tcagtgaagaatggtgctggatttc

SEQ ID NO: 151 - DNA, 25 bp, Artificial Sequence
  Description of Artificial Sequence Synthetic primer
agaaaagtttgtatagagggtggaa

SEQ ID NO: 152 - DNA, 25 bp, Artificial Sequence
  Description of Artificial Sequence Synthetic primer
tcaataaaaaataatactaaatttc

SEQ ID NO: 153 - DNA, 25 bp, Artificial Sequence
  Description of Artificial Sequence Synthetic primer
tcagtcttataaaaaggaaggttct

SEQ ID NO: 154 - DNA, 25 bp, Artificial Sequence
  Description of Artificial Sequence Synthetic primer
tgcttgtgggaaagctggaatatcc

SEQ ID NO: 155 - DNA, 25 bp, Artificial Sequence
  Description of Artificial Sequence Synthetic primer
ttagttttataaaaaggaaggtttt

SEQ ID NO: 156 - DNA, 25 bp, Artificial Sequence
  Description of Artificial Sequence Synthetic primer
tacttataaaaaaactaaaatatcc

SEQ ID NO: 157 - DNA, 21 bp, Artificial Sequence
  Description of Artificial Sequence Synthetic primer
atcagacaggagagtggggac

SEQ ID NO: 158 - DNA, 25 bp, Artificial Sequence
  Description of Artificial Sequence Synthetic primer
agctccacagccagacgccctcaga

SEQ ID NO: 159 - DNA, 21 bp, Artificial Sequence
  Description of Artificial Sequence Synthetic primer
attagataggagagtggggat

SEQ ID NO: 160 - DNA, 25 bp, Artificial Sequence
  Description of Artificial Sequence Synthetic primer
aactccacaaccaaacrccctcaaa

SEQ ID NO: 161 - DNA, 25 bp, Artificial Sequence
  Description of Artificial Sequence Synthetic primer
gggccctggagcctccctctgagaa

SEQ ID NO: 162 - DNA, 25 bp, Artificial Sequence
  Description of Artificial Sequence Synthetic primer
agcagtaggtcgcactggaagcccc

SEQ ID NO: 163 - DNA, 25 bp, Artificial Sequence
  Description of Artificial Sequence Synthetic primer
gggttttggagtttttttttgagaa

SEQ ID NO: 164 - DNA, 25 bp, Artificial Sequence
  Description of Artificial Sequence Synthetic primer
aacaataaatcrcactaaaaacccc

SEQ ID NO: 165 - DNA, 25 bp, Artificial Sequence
  Description of Artificial Sequence Synthetic primer
tgggggcttggctcacttccccaga

SEQ ID NO: 166 - DNA, 25 bp, Artificial Sequence
  Description of Artificial Sequence Synthetic primer
ggactttcaaaaaatttccactttg

SEQ ID NO: 167 - DNA, 25 bp, Artificial Sequence
  Description of Artificial Sequence Synthetic primer
tgggggtttggtttatttttttaga

SEQ ID NO: 168 - DNA, 25 bp, Artificial Sequence
  Description of Artificial Sequence Synthetic primer
aaactttcaaaaaatttccacttta

SEQ ID NO: 169 - DNA, 25 bp, Artificial Sequence
  Description of Artificial Sequence Synthetic primer
aaagagcagatttaagggaaagagg

SEQ ID NO: 170 - DNA, 24 bp, Artificial Sequence
  Description of Artificial Sequence Synthetic primer
ccaccacacacacacccccttcct

SEQ ID NO: 171 - DNA, 25 bp, Artificial Sequence
  Description of Artificial Sequence Synthetic primer
aaagagtagatttaagggaaagagg

SEQ ID NO: 172 - DNA, 24 bp, Artificial Sequence
  Description of Artificial Sequence Synthetic primer
ccaccacacacacacccccttcct

SEQ ID NO: 173 - DNA, 25 bp, Artificial Sequence
  Description of Artificial Sequence Synthetic primer
tttagtactagtaactgtcaaaggc

SEQ ID NO: 174 - DNA, 24 bp, Artificial Sequence
  Description of Artificial Sequence Synthetic primer
ggcggctggctggtaggtgatagt

SEQ ID NO: 175 - DNA, 25 bp, Artificial Sequence
  Description of Artificial Sequence Synthetic primer
tttagtattagtaattgttaaaggt

SEQ ID NO: 176 - DNA, 24 bp, Artificial Sequence
  Description of Artificial Sequence Synthetic primer
aacaactaactaataaataataat

SEQ ID NO: 177 - DNA, 23 bp, Artificial Sequence
  Description of Artificial Sequence Synthetic primer
ggaaggggagagtagtgaagggg

SEQ ID NO: 178 - DNA, 24 bp, Artificial Sequence
  Description of Artificial Sequence Synthetic primer
ccttggtcaccactccagtcacca

SEQ ID NO: 179 - DNA, 23 bp, Artificial Sequence
  Description of Artificial Sequence Synthetic primer
ggaaggggagagtagtgaagggg

SEQ ID NO: 180 - DNA, 24 bp, Artificial Sequence
  Description of Artificial Sequence Synthetic primer
ccttaatcaccactccagtcacca

SEQ ID NO: 181 - DNA, 25 bp, Artificial Sequence
  Description of Artificial Sequence Synthetic primer
ccactctcaagagaggcctgaacag

SEQ ID NO: 182 - DNA, 25 bp, Artificial Sequence
  Description of Artificial Sequence Synthetic primer
acctaaaatctcctcctgctcctgc

SEQ ID NO: 183 - DNA, 25 bp, Artificial Sequence
  Description of Artificial Sequence Synthetic primer
ttatttttaagagaggtttgaatag

SEQ ID NO: 184 - DNA, 25 bp, Artificial Sequence
  Description of Artificial Sequence Synthetic primer
acctaaaatctcctcctactcctac

SEQ ID NO: 185 - DNA, 25 bp, Artificial Sequence
  Description of Artificial Sequence Synthetic primer
ccctgggaaggggagaagatcaaga

SEQ ID NO: 186 - DNA, 25 bp, Artificial Sequence
  Description of Artificial Sequence Synthetic primer
ccctctctccctgccctctccaggc

SEQ ID NO: 187 - DNA, 25 bp, Artificial Sequence
  Description of Artificial Sequence Synthetic primer
ttttgggaaggggagaagattaaga

SEQ ID NO: 188 - DNA, 25 bp, Artificial Sequence
  Description of Artificial Sequence Synthetic primer
ccctctctccctaccctctccaaac

SEQ ID NO: 189 - DNA, 27 bp, Artificial Sequence
  Description of Artificial Sequence Synthetic primer
ggaaaaagcagcctagtacaagaagct

SEQ ID NO: 190 - DNA, 25 bp, Artificial Sequence
  Description of Artificial Sequence Synthetic primer
ctcttggccttggatttctggctgc

SEQ ID NO: 191 - DNA, 27 bp, Artificial Sequence
  Description of Artificial Sequence Synthetic primer
ggaaaaagtagtttagtataagaagtt

SEQ ID NO: 192 - DNA, 25 bp, Artificial Sequence
  Description of Artificial Sequence Synthetic primer
ctcttaaccttaaatttctaactac

SEQ ID NO: 193 - DNA, 27 bp, Artificial Sequence
  Description of Artificial Sequence Synthetic primer
agtctccagctttgaaaactgggcagg

SEQ ID NO: 194 - DNA, 27 bp, Artificial Sequence
  Description of Artificial Sequence Synthetic primer
cctccaaatcaccaagctggtctgagc

SEQ ID NO: 195 - DNA, 27 bp, Artificial Sequence
  Description of Artificial Sequence Synthetic primer
agtttttagttttgaaaattgggtagg

SEQ ID NO: 196 - DNA, 27 bp, Artificial Sequence
  Description of Artificial Sequence Synthetic primer
cctccaaatcaccaaactaatctaaac

SEQ ID NO: 197 - DNA, 26 bp, Artificial Sequence
  Description of Artificial Sequence Synthetic primer
tgtagttttcggagttagtgtcgcgc

SEQ ID NO: 198 - DNA, 25 bp, Artificial Sequence
  Description of Artificial Sequence Synthetic primer
cctacgatcgaaaacgacgcgaacg

SEQ ID NO: 199 - DNA, 30 bp, Artificial Sequence
  Description of Artificial Sequence Synthetic primer
gttttgtagtttttggagttagtgttgtgt

SEQ ID NO: 200 - DNA, 30 bp, Artificial Sequence
  Description of Artificial Sequence Synthetic primer
ctcaacctacaatcaaaaacaacacaaaca

SEQ ID NO: 201 - DNA, 20 bp, Artificial Sequence
  Description of Artificial Sequence Synthetic primer
cacacgcgcgaaaacaatat

SEQ ID NO: 202 - DNA, 20 bp, Artificial Sequence
  Description of Artificial Sequence Synthetic primer
tatgtatgttacgttcgtcg

SEQ ID NO: 203 - DNA, 25 bp, Artificial Sequence
  Description of Artificial Sequence Synthetic primer
cacacaaaaacaatatctcatttct

SEQ ID NO: 204 - DNA, 25 bp, Artificial Sequence
  Description of Artificial Sequence Synthetic primer
ttttttatgtatgttatgtttgttg

SEQ ID NO: 205 - DNA, 18 bp, Artificial Sequence
  Description of Artificial Sequence Synthetic primer
gggttttgcgagagcgcg

SEQ ID NO: 206 - DNA, 18 bp, Artificial Sequence
  Description of Artificial Sequence Synthetic primer
gctaacaaacgcgaaccg

SEQ ID NO: 207 - DNA, 24 bp, Artificial Sequence
  Description of Artificial Sequence Synthetic primer
ggggttttgtgagagtgtgtttag

SEQ ID NO: 208 - DNA, 26 bp, Artificial Sequence
  Description of Artificial Sequence Synthetic primer
taaacactaacaaacacaaaccaaac

SEQ ID NO: 209 - DNA, 23 bp, Artificial Sequence
  Description of Artificial Sequence Synthetic primer
tggtagtttttatgaaaggcgtc

SEQ ID NO: 210 - DNA, 19 bp, Artificial Sequence
  Description of Artificial Sequence Synthetic primer
cctctaaccgcccaccacg

SEQ ID NO: 211 - DNA, 24 bp, Artificial Sequence
  Description of Artificial Sequence Synthetic primer
atggtagtttttatgaaaggtgtt

SEQ ID NO: 212 - DNA, 20 bp, Artificial Sequence
  Description of Artificial Sequence Synthetic primer
ccctctaaccacccaccaca

SEQ ID NO: 213 - DNA, 24 bp, Artificial Sequence
  Description of Artificial Sequence Synthetic primer
ttattagagggtggggcggatcgc

SEQ ID NO: 214 - DNA, 21 bp, Artificial Sequence
  Description of Artificial Sequence Synthetic primer
gaccccgaaccgcgaccgtaa

SEQ ID NO: 215 - DNA, 24 bp, Artificial Sequence
  Description of Artificial Sequence Synthetic primer
ttattagagggtggggtggattgt

SEQ ID NO: 216 - DNA, 22 bp, Artificial Sequence
  Description of Artificial Sequence Synthetic primer
caaccccaaaccacaaccataa

SEQ ID NO: 217 - DNA, 25 bp, Artificial Sequence
  Description of Artificial Sequence Synthetic primer
ggttttttcggttagttgcgcggcg

SEQ ID NO: 218 - DNA, 24 bp, Artificial Sequence
  Description of Artificial Sequence Synthetic primer
ccaacgaaaacctcgcgacctccg

SEQ ID NO: 219 - DNA, 36 bp, Artificial Sequence
  Description of Artificial Sequence Synthetic primer
aaagagggaaaggtttttttggttagttgtgtggtg

SEQ ID NO: 220 - DNA, 29 bp, Artificial Sequence
  Description of Artificial Sequence Synthetic primer
aaactccaacaaaaacctcacaacctcca

SEQ ID NO: 221 - DNA, 22 bp, Artificial Sequence
  Description of Artificial Sequence Synthetic primer
gtggtggttattgggcgacggc

SEQ ID NO: 222 - DNA, 21 bp, Artificial Sequence
  Description of Artificial Sequence Synthetic primer
gctatttcgccgaccccgacg

SEQ ID NO: 223 - DNA, 21 bp, Artificial Sequence
  Description of Artificial Sequence Synthetic primer
tggtggttattgggtgatggt

SEQ ID NO: 224 - DNA, 21 bp, Artificial Sequence
  Description of Artificial Sequence Synthetic primer
actatttcaccaaccccaaga

SEQ ID NO: 225 - DNA, 21 bp, Artificial Sequence
  Description of Artificial Sequence Synthetic primer
tgcgagcgtttttttaaatgc

SEQ ID NO: 226 - DNA, 20 bp, Artificial Sequence
  Description of Artificial Sequence Synthetic primer
taaactcgcaaaccaactcg

SEQ ID NO: 227 - DNA, 23 bp, Artificial Sequence
  Description of Artificial Sequence Synthetic primer
gtttggttgttgtttattggttg

SEQ ID NO: 228 - DNA, 22 bp, Artificial Sequence
  Description of Artificial Sequence Synthetic primer
cctaaactcacaaaccaactca

SEQ ID NO: 229 - DNA, 27 bp, Artificial Sequence
  Description of Artificial Sequence Synthetic primer
cacaatttacaacgcgaaaacgacgaa

SEQ ID NO: 230 - DNA, 26 bp, Artificial Sequence
  Description of Artificial Sequence Synthetic primer
ttttgttttaggcggttcgggtgttc

SEQ ID NO: 231 - DNA, 31 bp, Artificial Sequence
  Description of Artificial Sequence Synthetic primer
cttacacaatttacaacacaaaaacaacaaa

SEQ ID NO: 232 - DNA, 29 bp, Artificial Sequence
  Description of Artificial Sequence Synthetic primer
gttttttgttttaggtggtttgggtgttt

SEQ ID NO: 233 - DNA, 29 bp, Artificial Sequence
  Description of Artificial Sequence Synthetic primer
taaaaaaccctgcccccaccgggcttacg

SEQ ID NO: 234 - DNA, 29 bp, Artificial Sequence
  Description of Artificial Sequence Synthetic primer
agtcgtatgacaattggtcgctaaccgag

SEQ ID NO: 235 - DNA, 25 bp, Artificial Sequence
  Description of Artificial Sequence Synthetic primer
aaaattttgtttttatcgggtttac

SEQ ID NO: 236 - DNA, 24 bp, Artificial Sequence
  Description of Artificial Sequence Synthetic primer
cgtataacaattaatcgctaaccg

SEQ ID NO: 237 - DNA, 27 bp, Artificial Sequence
  Description of Artificial Sequence Synthetic primer
aaaaaattttgtttttattgggtttat

SEQ ID NO: 238 - DNA, 27 bp, Artificial Sequence
  Description of Artificial Sequence Synthetic primer
aatcatataacaattaatcactaacca

SEQ ID NO: 239 - DNA, 29 bp, Artificial Sequence
  Description of Artificial Sequence Synthetic primer
tggggagctgagggcaagtcgcgcccgcc

SEQ ID NO: 240 - DNA, 27 bp, Artificial Sequence
  Description of Artificial Sequence Synthetic primer
gctgcctgatccctggccaccgtccgt

SEQ ID NO: 241 - DNA, 23 bp, Artificial Sequence
  Description of Artificial Sequence Synthetic primer
ggagttgagggtaagtcgcgttc

SEQ ID NO: 242 - DNA, 23 bp, Artificial Sequence
  Description of Artificial Sequence Synthetic primer
acctaatccctaaccaccgtccg

SEQ ID NO: 243 - DNA, 25 bp, Artificial Sequence
  Description of Artificial Sequence Synthetic primer
ggggagttgagggtaagttgtgttt

SEQ ID NO: 244 - DNA, 25 bp, Artificial Sequence
  Description of Artificial Sequence Synthetic primer
ctacctaatccctaaccaccatcca

SEQ ID NO: 245 - DNA, 29 bp, Artificial Sequence
  Description of Artificial Sequence Synthetic primer
gacccgcagaggcctggcagcgccgcgtt

SEQ ID NO: 246 - DNA, 26 bp, Artificial Sequence
  Description of Artificial Sequence Synthetic primer
tacaagcgtacgccaccacgcccggc

SEQ ID NO: 247 - DNA, 21 bp, Artificial Sequence
  Description of Artificial Sequence Synthetic primer
gtagaggtttggtagcgtcgc

SEQ ID NO: 248 - DNA, 17 bp, Artificial Sequence
  Description of Artificial Sequence Synthetic primer
gtacgccaccacgcccg

SEQ ID NO: 249 - DNA, 24 bp, Artificial Sequence
  Description of Artificial Sequence Synthetic primer
tttgtagaggtttggtagtgttgt

SEQ ID NO: 250 - DNA, 20 bp, Artificial Sequence
  Description of Artificial Sequence Synthetic primer
aacatacaccaccacaccca

SEQ ID NO: 251 - DNA, 29 bp, Artificial Sequence
  Description of Artificial Sequence Synthetic primer
tagggaagctggcgaggccgagccccgcc

SEQ ID NO: 252 - DNA, 28 bp, Artificial Sequence
  Description of Artificial Sequence Synthetic primer
ctcggcccgccacaacccgaaaggcgcc

SEQ ID NO: 253 - DNA, 22 bp, Artificial Sequence
  Description of Artificial Sequence Synthetic primer
gaagttggcgaggtcgagtttc

SEQ ID NO: 254 - DNA, 22 bp, Artificial Sequence
  Description of Artificial Sequence Synthetic primer
acccgccacaacccgaaaaacg

SEQ ID NO: 255 - DNA, 24 bp, Artificial Sequence
  Description of Artificial Sequence Synthetic primer
gggaagttggtgaggttgagtttt

SEQ ID NO: 256 - DNA, 24 bp, Artificial Sequence
  Description of Artificial Sequence Synthetic primer
caacccaccacaacccaaaaaaca

SEQ ID NO: 257 - DNA, 26 bp, Artificial Sequence
  Description of Artificial Sequence Synthetic primer
gtgaggtggccggcgccccggctggc

SEQ ID NO: 258 - DNA, 28 bp, Artificial Sequence
  Description of Artificial Sequence Synthetic primer
cccaccaggctccctctccgcccccgcc

SEQ ID NO: 259 - DNA, 22 bp, Artificial Sequence
  Description of Artificial Sequence Synthetic primer
aggtggtcggcgtttcggttgg

SEQ ID NO: 260 - DNA, 22 bp, Artificial Sequence
  Description of Artificial Sequence Synthetic primer
ccaaactccctctccgcccccg

SEQ ID NO: 261 - DNA, 24 bp, Artificial Sequence
  Description of Artificial Sequence Synthetic primer
tgaggtggttggtgttttggttgg

SEQ ID NO: 262 - DNA, 24 bp, Artificial Sequence
  Description of Artificial Sequence Synthetic primer
caccaaactcctcctccaccccca

SEQ ID NO: 263 - DNA, 23 bp, Artificial Sequence
  Description of Artificial Sequence Synthetic primer
gccaggggcgggcggcggggcgg

SEQ ID NO: 264 - DNA, 25 bp, Artificial Sequence
  Description of Artificial Sequence Synthetic primer
cgctgcatctccgctctgcgcccgc

SEQ ID NO: 265 - DNA, 17 bp, Artificial Sequence
  Description of Artificial Sequence Synthetic primer
ggggcgggcggcggggc

SEQ ID NO: 266 - DNA, 20 bp, Artificial Sequence
  Description of Artificial Sequence Synthetic primer
acatctccgctctacgcccg

SEQ ID NO: 267 - DNA, 20 bp, Artificial Sequence
  Description of Artificial Sequence Synthetic primer
ttaggggtgggtggtggggt

SEQ ID NO: 268 - DNA, 22 bp, Artificial Sequence
  Description of Artificial Sequence Synthetic primer
ctacatctccactctacaccca

SEQ ID NO: 269 - DNA, 29 bp, Artificial Sequence
  Description of Artificial Sequence Synthetic primer
cccgtccgcggcggggccgcggacgcgcc

SEQ ID NO: 270 - DNA, 22 bp, Artificial Sequence
  Description of Artificial Sequence Synthetic primer
ggggccgccgaggccagcagcc

SEQ ID NO: 271 - DNA, 22 bp, Artificial Sequence
  Description of Artificial Sequence Synthetic primer
tcgcggcggggtcgcggacgcg

SEQ ID NO: 272 - DNA, 22 bp, Artificial Sequence
  Description of Artificial Sequence Synthetic primer
aaaaccrccraaaccaacaacc

SEQ ID NO: 273 - DNA, 24 bp, Artificial Sequence
  Description of Artificial Sequence Synthetic primer
gtttgtggtggggttgtggatgtg

SEQ ID NO: 274 - DNA, 22 bp, Artificial Sequence
  Description of Artificial Sequence Synthetic primer
aaaaccrccraaaccaacaacc

SEQ ID NO: 275 - DNA, 26 bp, Artificial Sequence
  Description of Artificial Sequence Synthetic primer
gctgcccgcgtttgcgcagccccggg

SEQ ID NO: 276 - DNA, 28 bp, Artificial Sequence
  Description of Artificial Sequence Synthetic primer
gggaaggtcccacgggcgacaagacgct

SEQ ID NO: 277 - DNA, 22 bp, Artificial Sequence
  Description of Artificial Sequence Synthetic primer
ttgttygygtttgcgtagtttc

SEQ ID NO: 278 - DNA, 22 bp, Artificial Sequence
  Description of Artificial Sequence Synthetic primer
aaatcccacraacgacaaaacg

SEQ ID NO: 279 - DNA, 23 bp, Artificial Sequence
  Description of Artificial Sequence Synthetic primer
gttgttygygtttgtgtagtttt

SEQ ID NO: 280 - DNA, 24 bp, Artificial Sequence
  Description of Artificial Sequence Synthetic primer
aaaaatcccacraacaacaaaaca

SEQ ID NO: 281 - DNA, 27 bp, Artificial Sequence
  Description of Artificial Sequence Synthetic primer
gggggcataaaagccgcggccgcgcgg

SEQ ID NO: 282 - DNA, 30 bp, Artificial Sequence
  Description of Artificial Sequence Synthetic primer
gcagagaaatttccgcggctgggcgccggg

SEQ ID NO: 283 - DNA, 21 bp, Artificial Sequence
  Description of Artificial Sequence Synthetic primer
gtataaaagtcgcggtcgcgc

SEQ ID NO: 284 - DNA, 22 bp, Artificial Sequence
  Description of Artificial Sequence Synthetic primer
aaatttccgcgactaaacgccg

SEQ ID NO: 285 - DNA, 23 bp, Artificial Sequence
  Description of Artificial Sequence Synthetic primer
gggtataaaagttgtggttgtgt

SEQ ID NO: 286 - DNA, 26 bp, Artificial Sequence
  Description of Artificial Sequence Synthetic primer
aaaaaaatttccacaactaaacacca

SEQ ID NO: 287 - DNA, 19 bp, Artificial Sequence
  Description of Artificial Sequence Synthetic primer
ccaggctacttctcccctc

SEQ ID NO: 288 - DNA, 20 bp, Artificial Sequence
  Description of Artificial Sequence Synthetic primer
ctgtccagttctcagccaca

SEQ ID NO: 289 - DNA, 19 bp, Artificial Sequence
  Description of Artificial Sequence Synthetic primer
aaaagctccaggtcccttc

SEQ ID NO: 290 - DNA, 20 bp, Artificial Sequence
  Description of Artificial Sequence Synthetic primer
agggtttcttgccaagatcc

SEQ ID NO: 291 - DNA, 23 bp, Artificial Sequence
  Description of Artificial Sequence Synthetic primer
aaggcagaaggagccacgagtgc

SEQ ID NO: 292 - DNA, 24 bp, Artificial Sequence
  Description of Artificial Sequence Synthetic primer
ggccattttggtgcagtgacccca

SEQ ID NO: 293 - DNA, 20 bp, Artificial Sequence
  Description of Artificial Sequence Synthetic primer
actttcccaatctgccctac

SEQ ID NO: 294 - DNA, 20 bp, Artificial Sequence
  Description of Artificial Sequence Synthetic primer
ttctggattgtagcagatca

SEQ ID NO: 295 - DNA, 22 bp, Artificial Sequence
  Description of Artificial Sequence Synthetic primer
tcactcaaagcctcctgcctat

SEQ ID NO: 296 - DNA, 22 bp, Artificial Sequence
  Description of Artificial Sequence Synthetic primer
cagtgccttcacatagtcatcc

SEQ ID NO: 297 - DNA, 23 bp, Artificial Sequence
  Description of Artificial Sequence Synthetic primer
cttcctaccctcaagtatgtccg

SEQ ID NO: 298 - DNA, 23 bp, Artificial Sequence
  Description of Artificial Sequence Synthetic primer
gaggtgggaggacaggagttctt

SEQ ID NO: 299 - DNA, 19 bp, Artificial Sequence
  Description of Artificial Sequence Synthetic primer
cacacggctcacattgcat

SEQ ID NO: 300 - DNA, 19 bp, Artificial Sequence
  Description of Artificial Sequence Synthetic primer
cacgaacagcaaagcgacc

SEQ ID NO: 301 - DNA, 25 bp, Artificial Sequence
  Description of Artificial Sequence Synthetic primer
ctctgcaaattaatttggtttctgt

SEQ ID NO: 302 - DNA, 25 bp, Artificial Sequence
  Description of Artificial Sequence Synthetic primer
acagaaaccaaattaatttgcagag

SEQ ID NO: 303 - DNA, 23 bp, Artificial Sequence
  Description of Artificial Sequence Synthetic primer
tggtgcgacctctgtggcgactt

SEQ ID NO: 304 - DNA, 21 bp, Artificial Sequence
  Description of Artificial Sequence Synthetic primer
tcctgcaaggagggtggcttc

SEQ ID NO: 305 - DNA, 25 bp, Artificial Sequence
  Description of Artificial Sequence Synthetic primer
ctgaccagctgcactcggggatttc

SEQ ID NO: 306 - DNA, 22 bp, Artificial Sequence
  Description of Artificial Sequence Synthetic primer
gccgccggttgctgctacatga

SEQ ID NO: 307 - DNA, 21 bp, Artificial Sequence
  Description of Artificial Sequence Synthetic primer
agccttcggctgactggctgg

SEQ ID NO: 308 - DNA, 22 bp, Artificial Sequence
  Description of Artificial Sequence Synthetic primer
ctgcccatcatcatgacctgga

SEQ ID NO: 309 - DNA, 23 bp, Artificial Sequence
  Description of Artificial Sequence Synthetic primer
taaacagacatttatttccagac

SEQ ID NO: 310 - DNA, 23 bp, Artificial Sequence
  Description of Artificial Sequence Synthetic primer
gaaagaaatagtcaatatgcttg

SEQ ID NO: 311 - DNA, 27 bp, Artificial Sequence
  Description of Artificial Sequence Synthetic primer
ttgtagttatcttagaagatagcatgg

SEQ ID NO: 312 - DNA, 25 bp, Artificial Sequence
  Description of Artificial Sequence Synthetic primer
acgggaattactattaacataagcg

SEQ ID NO: 313 - DNA, 18 bp, Artificial Sequence
  Description of Artificial Sequence Synthetic oligonucleotide
tttttttttttttttttt

(19) United States
(12) Patent Application Publication
(10) Pub. No.: US 2008/0249118 A1
(43) Pub. Date: Oct. 9, 2008

(54) Silencing of Tumor-Suppressive Genes by Cpg-Methylation in Prostate Cancer

(76) Inventors: Heiko Hermeking, Martinsried (DE); Dmitri Lodyguine, Munchen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/596,728

(22) PCT Filed: May 13, 2005
(86) PCT No.: PCT/EP05/05285

§ 371 Date: Nov. 16, 2006

(30) Foreign Application Priority Data
May 17, 2004 (EP) 04011680.8

(51) Int. Cl. A61K 31/513; C12Q 1/68; A61P 35/00; C40B 30/00

(52) U.S. Cl. 514/274; 435/6; 506/7

(57) ABSTRACT

The present invention relates to method and kits for diagnosing and/or treating prostate cancer. The method and kit relate to the determination and/or modulation of the methylation degree of tumor suppressive genes in biological samples. More particularly, CpG-methylation of 14-3-3σ was found in all primary PCa samples analysed, but not in matching normal prostate epithelial cells or benign prostate hyperplasia (BPH). CpG-methylation was accompanied by a decrease or loss of 14-3-3σ protein expression in primary PCa and PCa cell lines. PCa-precursor lesions, known as prostatic intraepithelial neoplasia (PIN), also displayed decreased levels of 14-3-3σ expression, whereas normal prostate epithelial cells and BPH showed high levels of 14-3-3σ protein expression. The generality of CpG-methylation in PCa suggests that silencing of 14-3-3σ significantly contributes to the formation of PCa. Furthermore, the CpG-methylation of 14-3-3σ in PCa may be exploited for diagnostic purposes.

The present invention relates to a method and kit for diagnosing and/or treating prostate cancer. The method and kit relate to the determination and/or modulation of the methylation degree of tumor suppressive genes in biological samples.

More particularly, CpG-methylation of several genes, e.g. 14-3-3σ was found in primary PCa samples analysed, but not in matching normal prostate epithelial cells or benign prostate hyperplasia (BPH). CpG-methylation was accompanied by a decrease or loss of 14-3-3σ protein expression in primary PCa and PCa cell lines. PCa-precursor lesions, known as prostatic intraepithelial neoplasia (PIN), also displayed decreased levels of 14-3-3σ expression, whereas normal prostate epithelial cells and BPH showed high levels of 14-3-3σ protein expression. The generality of CpG-methylation in PCa suggests that silencing of 14-3-3σ significantly contributes to the formation of PCa. Furthermore, the CpG-methylation of genes, e.g. 14-3-3σ in PCa may be exploited for diagnostic and/or therapeutic purposes.

Prostate cancer (PCa) is the most commonly diagnosed malignancy in the male population of the western world. For the year 2004 it is estimated that prostate cancer will be diagnosed in 230,110 men and cause death in 29,900 cases in the US¹. Small prostatic carcinomas are detected in 29% of men between 30 and 40 years of age and in 64% of men from 60 to 70 years of age². This usually indolent disease behaves aggressively in 25% of the affected men and accounts for ˜10% of all male cancer deaths, second only to lung cancer¹. The molecular basis of PCa is still poorly understood, particularly due to the extreme heterogeneity of primary tumors.

Genetic inactivation of tumor suppressor genes known from other types of cancer, is detected at a relatively low frequency in early stage PCa, e.g. mutations of p53, p16 and Rb occur in a minor fraction of PCa^{3,4,5}. PTEN may be the most consistently affected tumor suppressor gene in PCa: 30% of primary and 63% of metastatic PCas display mutation or deletion of PTEN⁶. The identification of PCa-specific tumor suppressor genes has been hindered by several factors. First, no known syndrome predisposing to PCa has been identified, which could serve to determine genetic loci harboring tumor suppressor genes. Second, the high prevalence of PCa makes it difficult to identify familial clustering and cases of early onset, although a role for genetic predisposition has been proposed for ˜40% of all PCa cases 3. At least seven chromosomal regions have been associated with a predisposition to PCa⁷. However, the candidate genes identified in these regions are altered at low frequencies, which suggests that genetic predisposition to PCa may involve alterations in additional genes. Polymorphisms in detoxifying or repair genes, as GSTT, GSTP1 and MTHFR may also contribute to predisposition for PCa⁸.

Silencing of tumor suppressor gene expression by CpG-methylation is presumably of equal importance for tumor development as functional inactivation by point mutation or allelic loss^{9,10}. For example in tumors with LOH at the p16/INK4A locus the remaining allele is inactivated by either point mutation or CpG-methylation. Aberrantly methylated CpG-dinucleotides are bound by the methyl-CpG binding proteins MBD1-4 and MeCP2, which recruit histone deacetylases (HDACs) to the respective promoters and thereby generate transcriptionally inactive chromatin. How the CpG-methylation of tumor suppressor genes is established during tumor progression is still unclear, but stochastic, age-associated accumulation of aberrantly methylated CpG-sites may be involved in this process¹⁰. Experimental reversion of CpG-methylation leads to re-expression of silenced genes in tumor cells, which may have profound consequences at the cellular level: e.g. restored sensitivity to apoptotic stimuli after reactivation of caspase-8¹¹ and Apaf-1¹² or inhibition of cell-proliferation after re-expression of the CDK-inhibitor p16¹³. Consistent with a role of gene repression mediated by CpG-methylation in tumorigenesis, inactivation of MBD2 suppresses intestinal tumorigenesis in Apc^{min} mice¹⁴. In prostate cancer, CpG-hypermethylation and subsequent loss of expression has been reported for a limited number of genes: e.g. GSTP1 is silenced in ˜90%, RASSF1A in ˜63% and RARβ2 in ˜79% of primary PCa¹⁵⁻¹⁷. CpG-methylation of GSTP1 has proven useful for diagnosis of PCa cells in biopsies and body fluids¹⁸.

Silencing of hypermethylated genes depends on both, CpG-methylation and subsequent recruitment of MBD-associated HDAC activity¹⁹. Therefore, optimal re-expression of genes silenced by CpG-methylation is detectable after simultaneous inhibition of DNA-methyltransferase and histone deacetylase activity by 5-aza-2′-deoxycytidine (5Aza-2′dC) and trichostatin A (TSA), respectively. The CpG-methylation pattern of human cancer cell lines closely reflects the pattern detected in primary tumors of the same tumor type²⁰. Therefore, comprehensive analysis of the gene expression after treatment of tumor cell lines with 5Aza-2′dC and TSA was recently used to identify genes specifically silenced in tumors^{21,22}. The major advantage of this approach is the functional link between de-methylation and re-expression of a particular gene, which other approaches, such as restriction landmark genomic scanning, differential methylation hybridization or CpG-methylation-specific RDA do not provide¹⁰.

In the present application, the identification of genes epigenetically silenced in Pca is described. Validation and characterisation of the CpG-methylation patterns in a significant number of primary PCa samples suggests that inactivation of these genes is involved in the development of PCa.

The frequencies of in vivo CpG-methylation of genes in primary PCa samples were: 14-3-3σ: 100%, GPX3: 93%, DDB2: 85%, SFRP1: 83%, COX2/PTGS2: 78%, HPGD: 71%, GSTM1: 58%, DKK3: 68% and KIP2/p57: 56%. These high frequencies in combination with the known functional properties of the respective gene-products, as genome protection (e.g. GSTM1, GPX3, DDB2), inhibition of cell cycle progression (e.g. p57, 14-3-3σ) and regulation of differentiation (e.g. DKK3, SFRP1), allow the conclusion that inactivation of the genes identified here significantly contributes to the formation of prostate carcinoma. Furthermore, detection and modulation of the CpG-methylation of these genes is useful for diagnostic and therapeutic purposes, particularly for the early detection and/or efficient therapy of prostate carcinoma.

A first aspect of the present invention relates to a method for diagnosing prostate carcinoma comprising determining of the methylation degree in the genomic locus of a gene selected from the group consisting of 14-3-3σ, DDB2, GPX3, GSTM1, SFRP1, DKK3, p57/KIP2, COX-2/PTGS2, HPGD and combinations thereof in a sample, wherein a hypermethylation is indicative for prostate cancer.

A further aspect of the invention relates to a kit comprising determining of the methylation degree in the genomic locus of a first gene selected from the group consisting of 14-3-3σ, SFRP1, COX2/PTGS2, GSTM1 and combinations thereof and in the genomic locus of a second gene selected from the group consisting of GPX3, DKK3 and combinations thereof.

The method and kit of the present invention preferably allow the determination of the methylation degree in a CpG sequence associated with a gene or combination of genes as indicated above. The methylation degree may be determined in the coding sequence of the above genes, or in regions located 5′ or 3′ to the coding region. More preferably, the methylation degree in the promoter region of the genes is determined.

The sample to be tested may be a prostate tissue section, preferably a section from a prostate lesion. In some embodiments of the invention it may be desired to enrich potential prostate carcinoma cells, from tissue samples. This enrichment may be carried out for example by laser microdissection.

Alternatively, the sample to be tested may be body fluid such as urine, blood, serum, plasma etc. The sample may be subjected to a treatment procedure which allows enrichment and/or isolation of genomic DNA from cells contained in the sample. The sample is preferably derived from a human subject, preferably a subject which is to be tested for PCa.

The determination of the methylation degree preferably occurs on the nucleic level, i.e. a direct determination of the methylation degree of a genomic nucleic acid is carried out. The methylation degree is determined in relation to the level of methylation in a reference genomic sequence such as a housekeeping gene or in the genomic locus of a corresponding gene derived from non-cancer tissue. Further, the methylation degree can be monitored in terms of a “quantitative increase”, e.g. by real time polymerase chain reaction (PCR). In a preferred embodiment, the determination may comprise bisulfite sequencing. Bisulfite sequencing encompasses treatment of genomic DNA with a bisulfite reagent which leads to a de-amination of unmethylated cytosine to thymidine residues, whereas methylated CpG residues are protected. Thus, the methylation degree may be determined by subsequent sequence analysis according to known procedures in the desired region.

The determination of the methylation degree may further comprise methylation-specific nucleic acid amplification, particularly methylation-specific PCR (MSP). This procedure involves the use of specific primer sequences which allow discrimination between methylated and unmethylated sequences.

The diagnostic method of the present invention may be carried out in any suitable test format. Preferably, however, the method comprises a microarray analysis. In this test formate, a nucleic acid array is provided comprising a plurality of different areas comprising nucleic acid hybridization probes capable of hybridizing with nucleic acid molecules to be detected. The nucleic acid molecules to be detected may be fragments of genomic DNA molecules optionally after treatment with bisulfite reagents or products from a sequence analysis and/or products from a methylation-specific nucleic acid amplification. In a preferred embodiment, the nucleic acids to be detected comprise labelling groups, e.g. chromophores and/or fluorescence groups. The microarray may be a planar structure or a microchannel device.

The method may comprise a qualitative and/or a quantitative determination of the methylation degree. A quantitative detection usually comprises the determination of labelling groups.

In a preferred embodiment of the invention, the determination of the methylation degree is carried out in the genomic locus of a first gene selected from the group consisting of 14-3-3σ, SFRP1, COX2/PTGS2, GSTM1 and combinations thereof and in the genomic locus of a second gene selected from the group consisting of GPX3, DKK3 and combinations thereof. Especially preferred combinations are combinations of 14-3-3σ with at least one of GPX3 and DKK3.

The kit for diagnosing prostate carcinoma comprises reagents for determining the methylation degree in the genomic locus of a gene or a combination of genes as listed above. The reagents may comprise nucleic acid amplification primers and/or hybridization probes. Further, the reagents may optionally comprise enzymes, nucleotides including chain termination nucleotides and labelling groups for sequencing reactions and/or enzymes, nucleotides and labelling groups for nucleic acid amplification reactions. Further, the reagents may comprise compounds for bisulfite treatment of nucleic acid samples.

The reagents may be present in form of solutions or dry products. Individual reagents may be present in separate containers. Alternatively, a plurality of reagents may be present in a single container. Further, the kit may contain user's instructions.

Still a further aspect of the invention relates to a method for treating prostate carcinoma comprising modulating, e.g. decreasing the methylation degree in the genomic locus of a gene selected from the group consisting of 14-3-3σ, DDB2, GPX3, GSTM1, SFRP1, DKK3, p57/KIP2, COX-2/PTGS2, HPGD and combinations thereof in a subject suffering from hypermethylation-associated prostate cancer. The subject is preferably a human patient.

The treatment preferably comprises administration of demethylating agents in an amount which is sufficient to provide an at least partial demethylation of the genes as indicated above.

Examples of demethylating agents used for genomic demethylation can be found in Table 1 below.

TABLE 1
Substances used for genomic demethylation.

| | | | |
|---|---|---|---|
| | Commercial | Cell | |
| Compound | name | culture | Clinical application |

Nucleoside analog inhibitors

| | | | | |
|---|---|---|---|---|
| 5-Azacytidine | Vidaza | 0.5-10 | μM | FDA approved for MDS |
| | | | | 75 mg/m²/day for 7 days every 28 |
| | | | | days, |
| 5-Aza- | Decitabine, | 0.5-10 | μM | PhaseI/II |
| 2′deoxycytidine | dacogen | | | 20-100 mg/m²/day |
| Arabinosyl-5- | Fazarabine | 25 | μM | PhaseI/II |
| azacytidine | | | | MTD: 425 mg/m²/hour infused over 72 |
| | | | | hours every 3 to 4 weeks |
| 5,6-dihydro-5- | DHAC | 1-10 | μM | PhaseI/II |
| azacytidine | | | | 1500 mg/m²/day |
| 5-Fluoro- | Gemcitabine | 1-30 | μM | PhaseI/II |
| 2′deoxycytidine | | | | 1000 mg/m²/day |
| 1-(beta-D- | Zebularine | 100 | μM | ND |
| ribofuranosyl)-1,2- | | | | |
| dihydropyrimidin-2- | | | | |
| one | | | | |

Non-nucleoside inhibitors

| | | | | |
|---|---|---|---|---|
| Epigallocatechin-3- | EGCG | 5-50 | μM | ND |
| gallate | | | | |
| Hydralazine | | 10 | μM | Used as cardiovascular drug |
| Procainamide | | 10 | μM | Used as cardiovascular drug |
| Procaine | | 500 | μM | Used as anesthetic drug |
| 30-mer GC-box | | 5-50 | nM | ND |
| oligonucleotides | | | | |
| Valproic acid | | 1-20 | mM | Used for treatment of epilepsy |

Repressors of DNMT expression

| | | | | |
|---|---|---|---|---|
| RNAi | | 25-100 | nM | ND |
| phosphorothioate | MG98 | 50-70 | nM | Phase II |
| antisense | | | | 360 mg/m²2 h infusion 2x a week for |
| oligodeoxynucleotide | | | | 3 weeks out of every 4 |

Alternatively, target-specific inactivation of the particular genes identified herein, as well as other genes involved in hypermethylation-associated tumors might be performed by the targeted, gene-specific removal of methyl groups. In particular, this might be achieved by target-specific RNAi approaches or the use of specific enzymes with demethylating activity. Generally, a target-specific approach would have less side effects than global demethylation.

Further, the present application shall be explained by the following Figures and Examples:

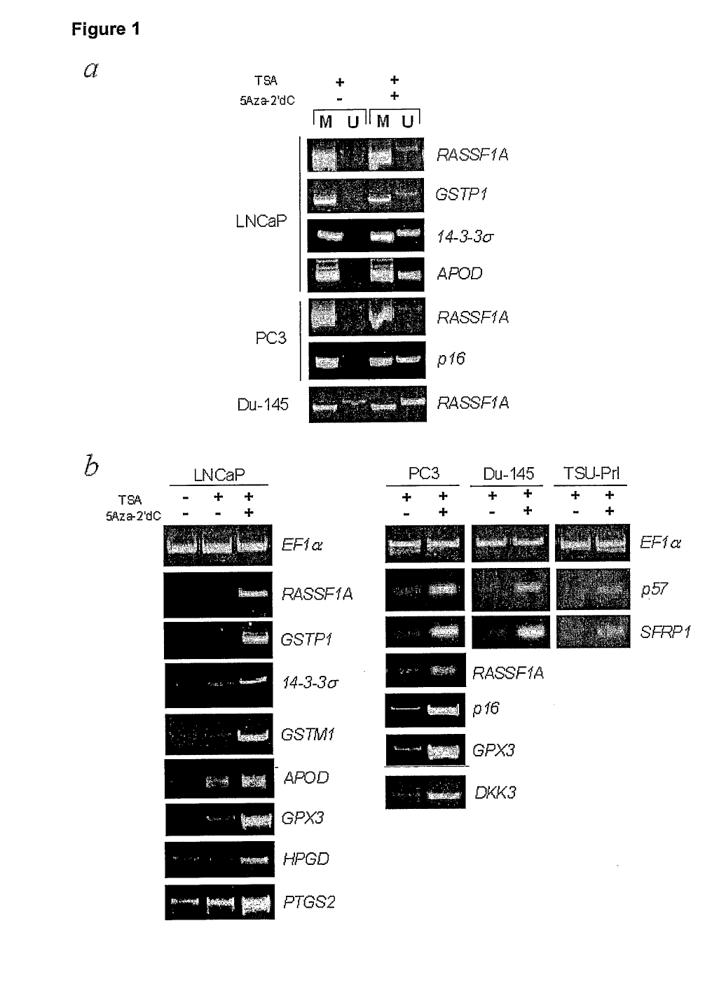

FIG. 1

Pharmacological reversion of epigenetic silencing in prostate carcinoma cell lines.

The PCa cell lines LNCaP, PC3 and Du-145 were treated with 5′-Aza-2′dC for 72 hours and with TSA for the last 24 hours, with TSA for 24 hours or left untreated.

a, After bisulfite treatment genomic DNA was subjected to MSP analysis with primers specific for the indicated genes (see also Table 2). The PCR-products labelled with “M” were generated by methylation-specific primers, and those labelled with “U” by primers specific for un-methylated DNA.

b, mRNA expression of the indicated genes was analysed by semi-quantitative RT-PCR. As a loading control and expression standard amplification of the house keeping gene EF1α was used. DNA concentrations were adjusted by quantitative real-time PCR (data not shown). For experimental details see Methods and legend of Table 2.

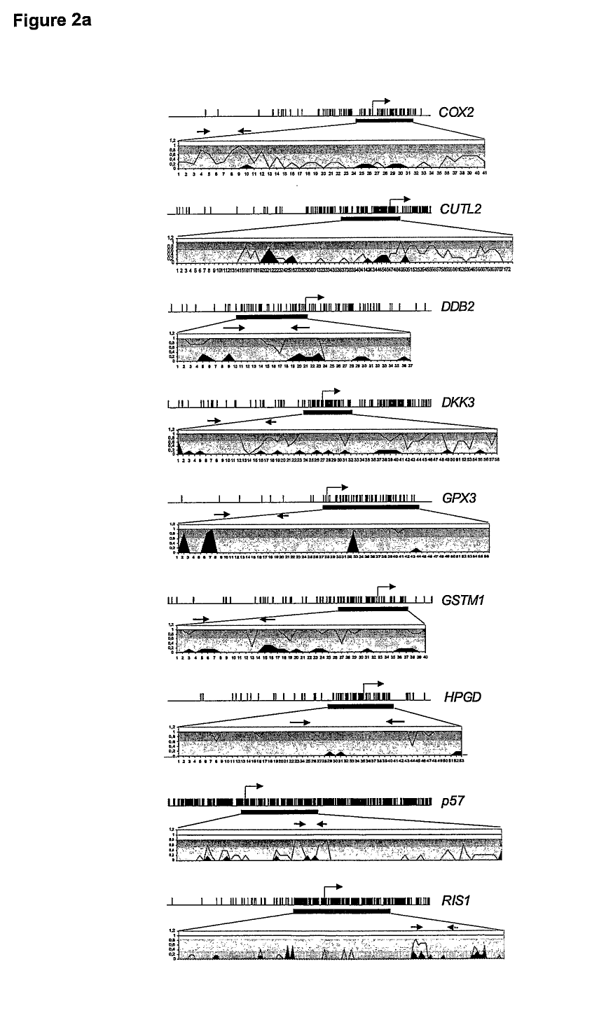

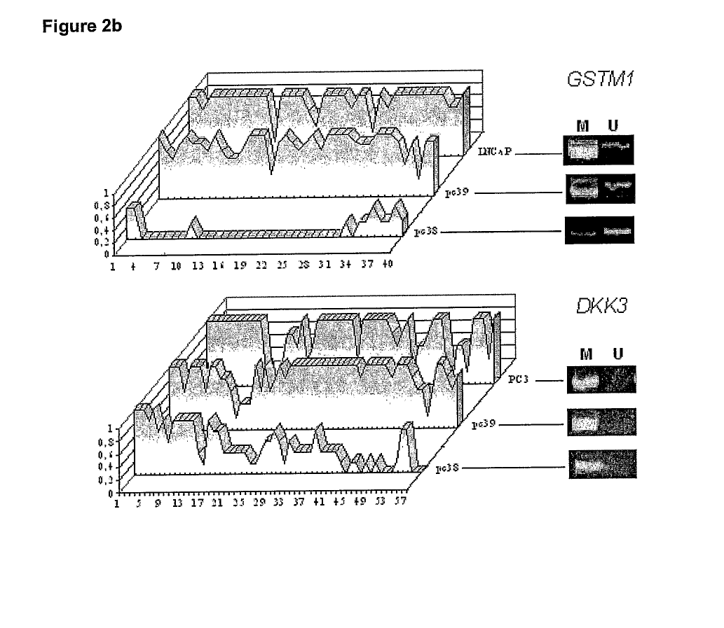

FIG. 2

Analysis of CpG-methylation patterns by bisulfite sequencing.

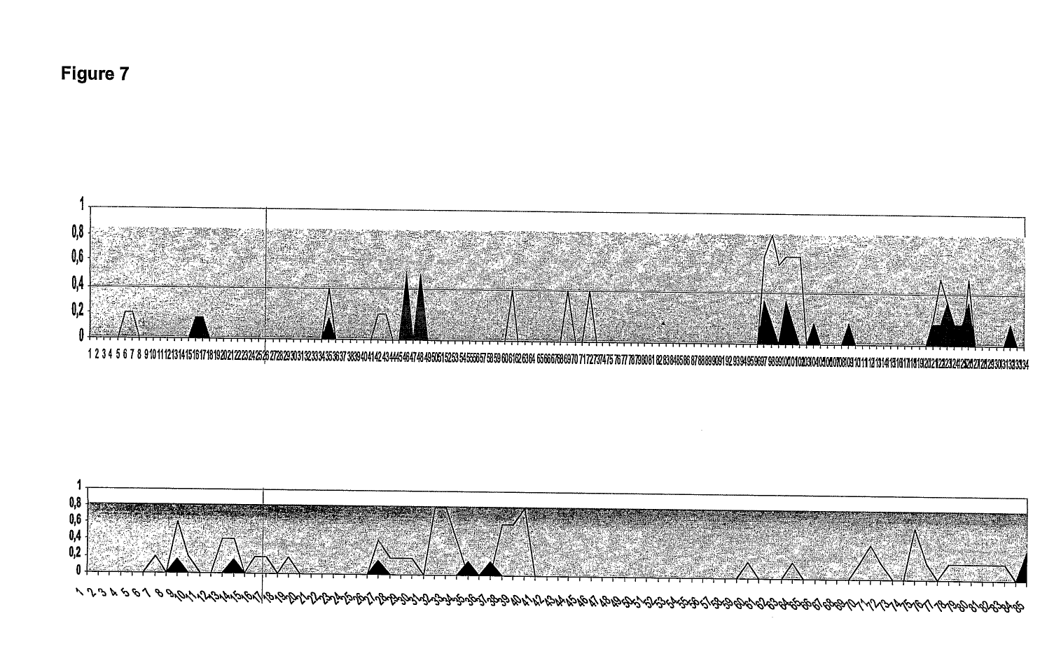

a, Schematic representation of the CpG-distribution and CpG-methylation in the promoter regions of the genes analysed by bisulfite sequencing. The depicted areas correspond to genomic DNA sequences of 2.5 kbp. Vertical bars represent CpG-dinucleotides. The position of the transcription start site is indicated by an arrow. Horizontal, black rectangles indicate areas which were amplified and subcloned after bisulfite treatment. Results of sequencing of at least 6 individual subclones for each area are shown enlarged: shaded chart areas represent frequencies of methylated CpG-dinucleotides within the respective fragments. The y-axis corresponds to the relative abundance of methylation of the CpG-dinucleotide at the indicated relative positions. The exact location of amplified fragments and CpG-dinucleotides is given in Table 2. For RIS1 and p57 results are provided at a larger scale in FIG. 7.

b. Determination of CpG methylation patterns of genes potentially silenced in PCa. Bisulfite sequencing was performed with genomic DNA derived from LNCaP (for COX2/PTGS2, DDB2, GSTM1 and HPGD genes), PC3 (CUTL2 DKK3, GPX3, and p57) and Du-145 (RIS1) cells or PrECs (each gene). CpG-distribution and CpG methylation are shown. The depicted areas correspond to genomic DNA sequences of 2.5 kbp. Vertical bars represent CpG-dinucleotides. The position of the transcription start site is indicated by an arrow. Horizontal, black rectangles indicate areas which were amplified and subcloned after bisulfite treatment. Results of sequencing of at least 6 individual subclones for each area are shown: grey shaded chart areas represent frequencies of methylated CpG-dinucleotides within the respective fragments in PCa cell lines. Black shaded areas show methylation pattern detected in PrECs. The y-axis corresponds to the relative abundance of methylation of the CpG-dinucleotide at the indicated relative positions. The exact location of amplified fragments and CpG-dinucleotides is given in Table 2.

c. Comparison of GSTM1 and DKK3 CpG-methylation pattern in cell lines and primary tumors. Tumor cells were isolated from paraffin-embedded tumor-sections using laser micro-dissection and gDNA was isolated. Results of a parallel MSP-analysis of the gDNAs employed for bisulfite sequencing is depicted. For experimental details see Methods.

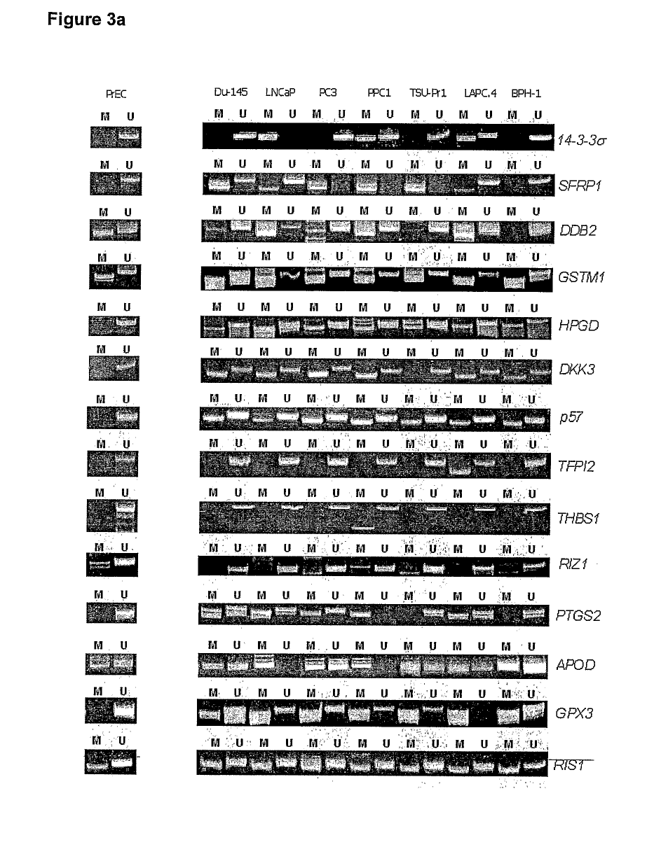

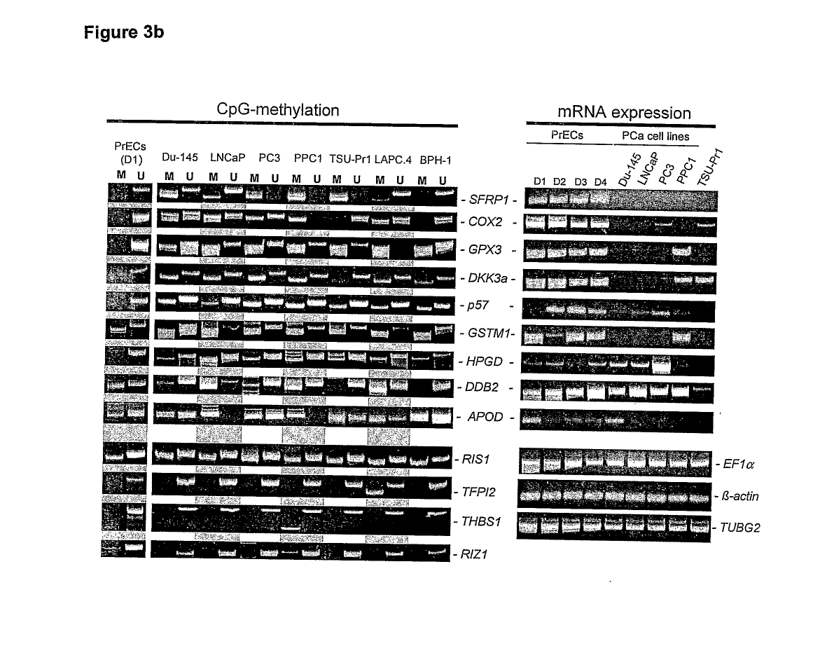

FIG. 3a

MSP analysis of a series of PCa cell lines and primary prostate epithelial cells.

Genomic DNA was isolated from exponentially proliferating cells, treated with sodium bisulfite and used as a template for MSP-analysis with two primer sets (M and U) for the indicated gene. BPH1=benign prostate hyperplasia cells immortalized with SV40 large T antigen. For detail see Methods and in the legend of Table 2.

FIG. 3b

Comparative analysis of gene expression.

RT-PCR analysis of the indicated genes in non-transformed and tumor cells. D1-D4 samples represent primary prostate epithelial cells (PrECs) from four different donors. β-actin and γ-tubulin (TUBG2) were used as additional standards.

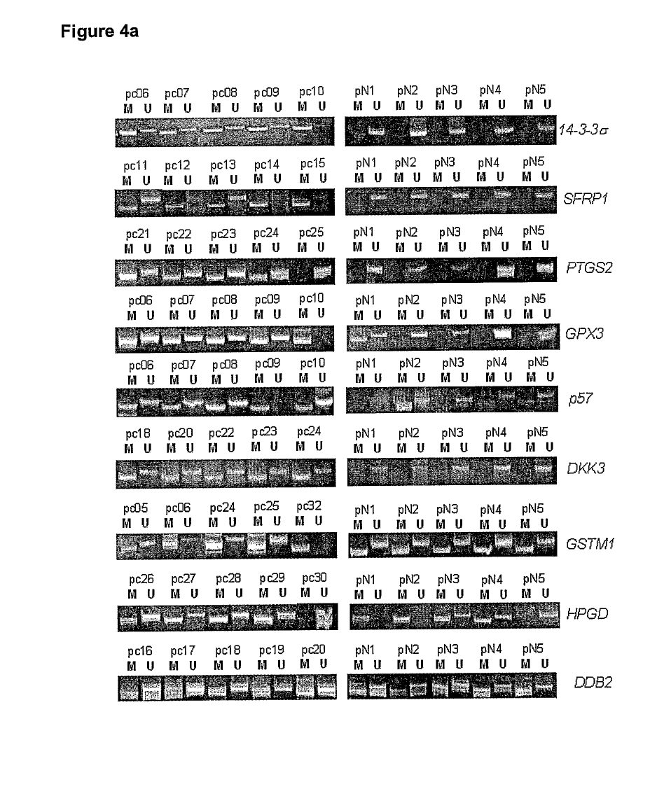

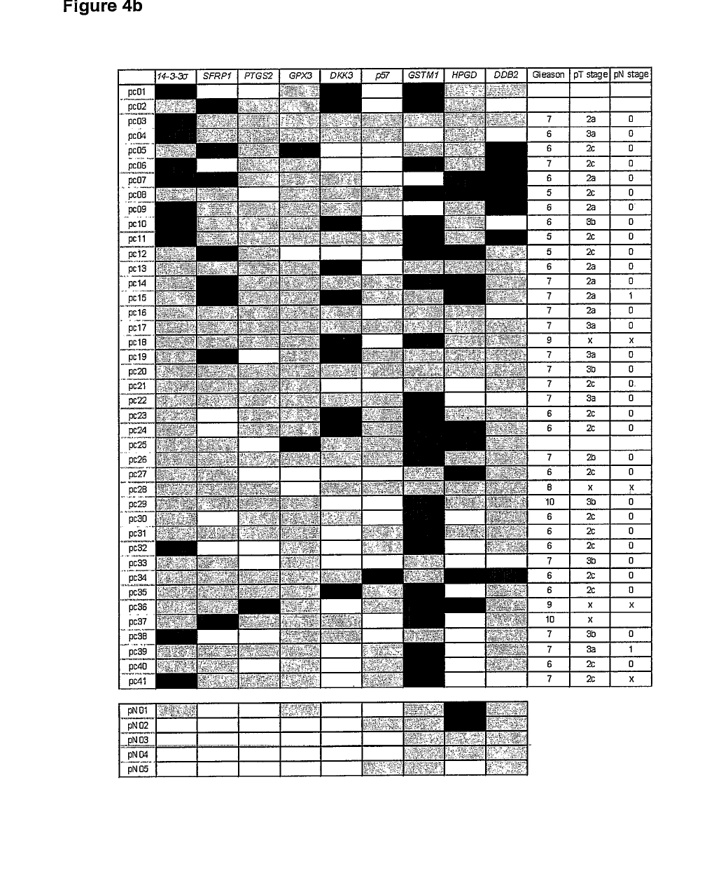

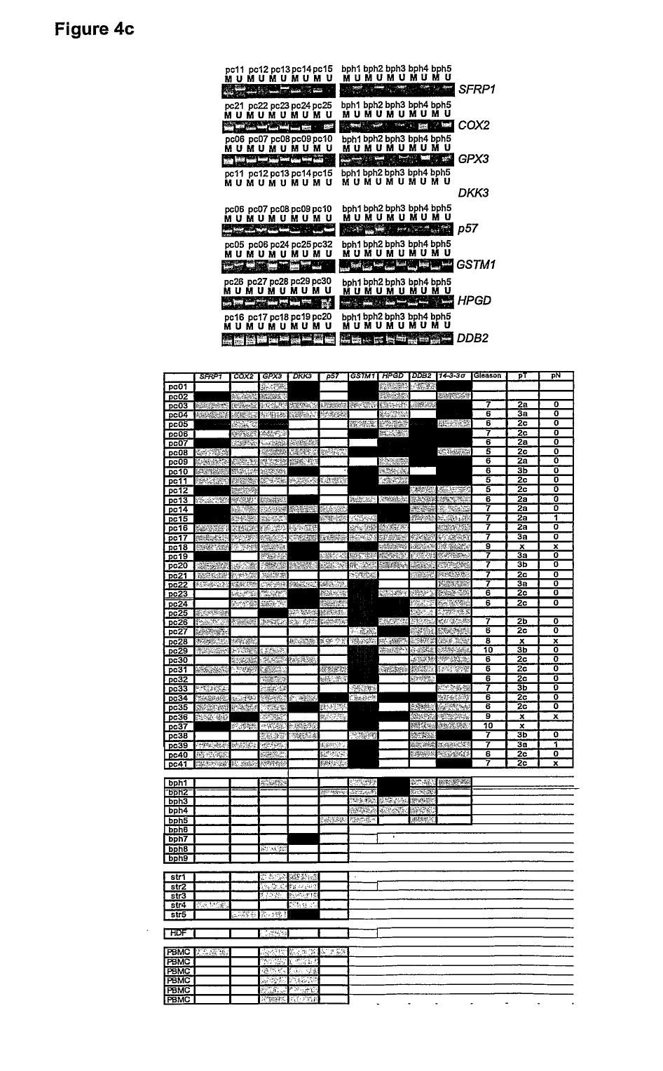

FIG. 4

MSP-analysis of in vivo CpG-methylation after laser-microdissection.

Tumor cells or normal prostate epithelial cells were isolated from paraffin-embedded tumor-sections derived from 41 different patients (pc01-pc41) using laser-pressure catapulting (see Methods for details).

a. Representative examples of MSP analysis of 5 tumor cell samples and 5 samples derived from normal epithelial cells for the indicated genes.

b. Summary of MSP results. Gene names are indicated on the top. Each row represents a primary PCa tumor or, in the lower part, a sample of primary prostate epithelial cells, isolated by laser-microdissection. Color coding: white=no CpG-methylation detected; grey=PCR-product indicative of CpG-methylation has roughly the same abundance as the PCR specific for un-methlyated allele; black=CpG-methylated allele dominant over un-methylated allele or un-methylated allele absent.

c. Summary of MSP results. Gene names are indicated on the top. Each row represents a primary PCa tumor (pc01-41), non-neoplastic prostate epithelial cells (bph1-9) or prostate stroma samples (str1-5) isolated by laser microdissection. Human diploid fibroblasts (HDF) derived from skin and PBMC (peripheral blood mononuclear cells) were cultured in vitro. The numbering is not meant to indicate that the different cell types were obtained from the same patient. Color coding: white=no significant CpG methylation detected; grey=PCR-product representing CpG methylation has a similar intensity as the PCR specific for un-methylated allele; black=allele with CpG methylation dominant over unmethylated allele or unmethylated allele absent.

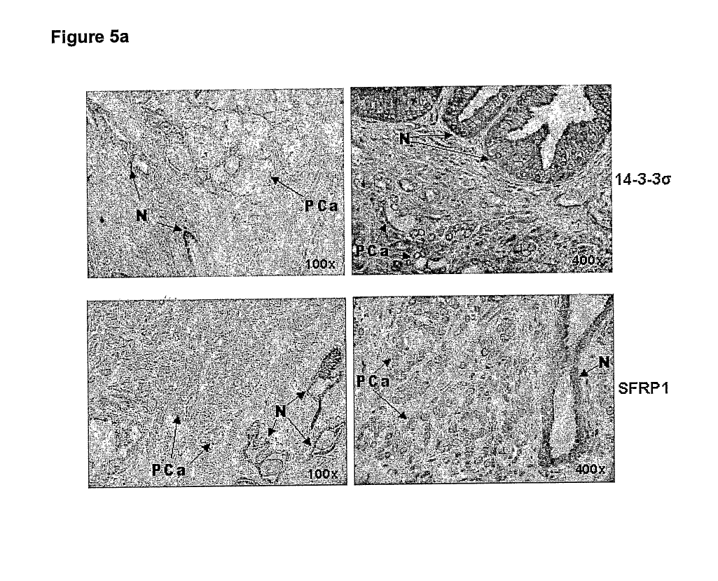

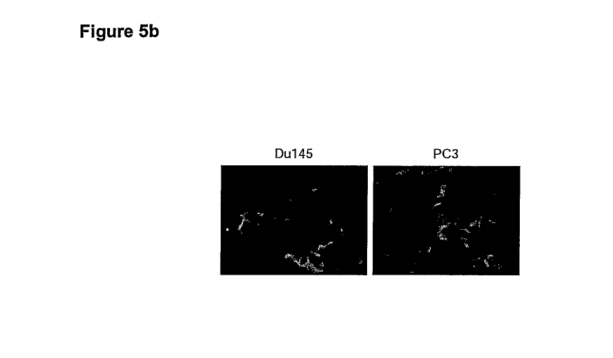

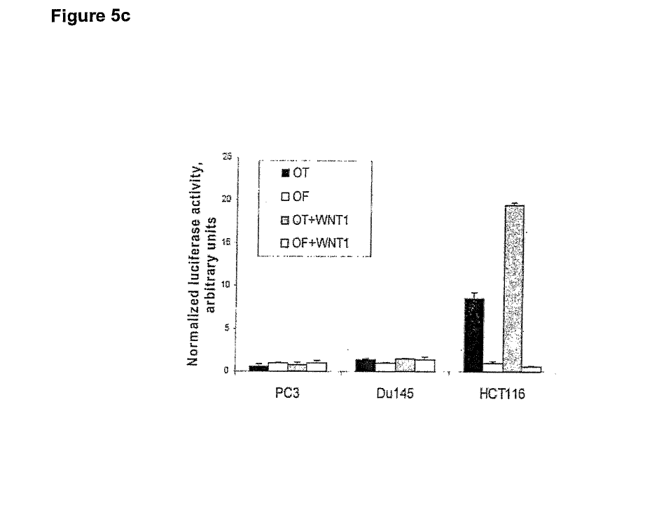

FIG. 5

Expression of proteins encoded by genes silenced in primary Pca and analysis of the β-catenin/TCF4 pathway.

a. An immunohistochemical detection of SFRP1 and 14-3-3σ (N: non-neoplastic prostate epithelial cells; PCa: prostate cancer cells) is shown. Left picture is taken at 100× magnification; right picture at 400× magnification.

b. Detection of β-catenin in PC3 and Du-145 Pca cells by immunofluoresence.

c. Analysis of TCF/LEF reporter activity in Pca (PC3, Du145) and colon (HCT116) cancer cell lines. Cells were transfected with a pGL3-OT (OT) TCF/LEF reporter construct, or with pGL3-OF (OF), a negative control containing a mutated TCF binding site. A WNT1 expression construct or its backbone construct were co-transfected. Luciferase activity was measured 36 hours after transfection. Transfection efficiency was normalized by co-transfection of a β-galactosidase encoding plasmid. The assays were performed in triplicates (standard deviation indicated). The values obtained for transfection of the OF-plasmid alone were set to one to visualize the differences among the three different cell lines.

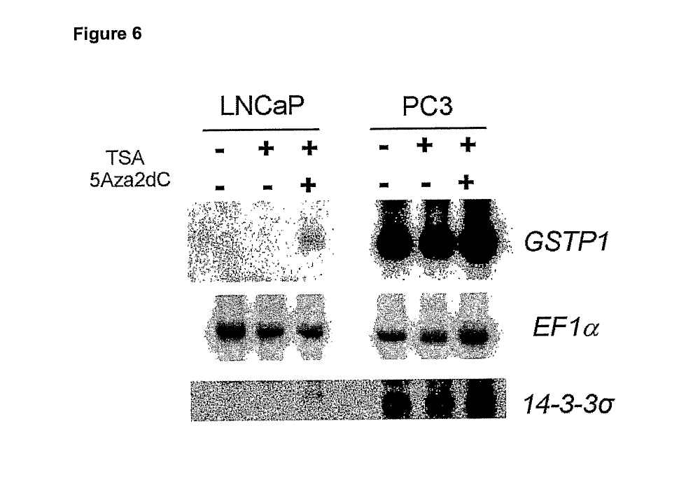

FIG. 6

De-methylation-dependent induction of GSTP1 and 14-3-3σ transcripts in LNCaP cell line detected by Northern blot hybridization. 15 μg of total RNA were resolved on a denaturing agarose gel, transferred onto a nitrocellulose membrane and hybridized in QuickHyb solution (Stratagene) with P³²-labeled probes specific for GSTP1, elongation factor 1σ or 14-3-3σ. The probes were directed against the 3′-UTR of the respective mRNAs.

FIG. 7

Large scale diagrams of the CpG-methylation pattern of RIS1 in the Du-145 cell line and of p57 in the cell line PC3. See legend of FIG. 2 for details.

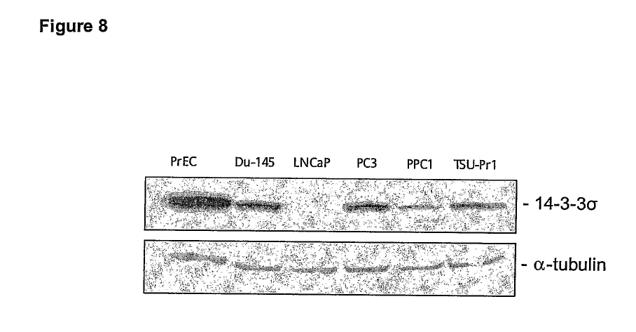

FIG. 8

Analysis of 14-3-3σ CpG-methylation and expression in prostate cancer cell lines.

Detection of 14-3-3σ protein expression. Western blot analysis was performed with extracts from exponentially growing cells using an affinity purified rabbit 14-3-3σ anti-serum as described previously⁴¹. Detection of α-tubulin was employed as a loading control.

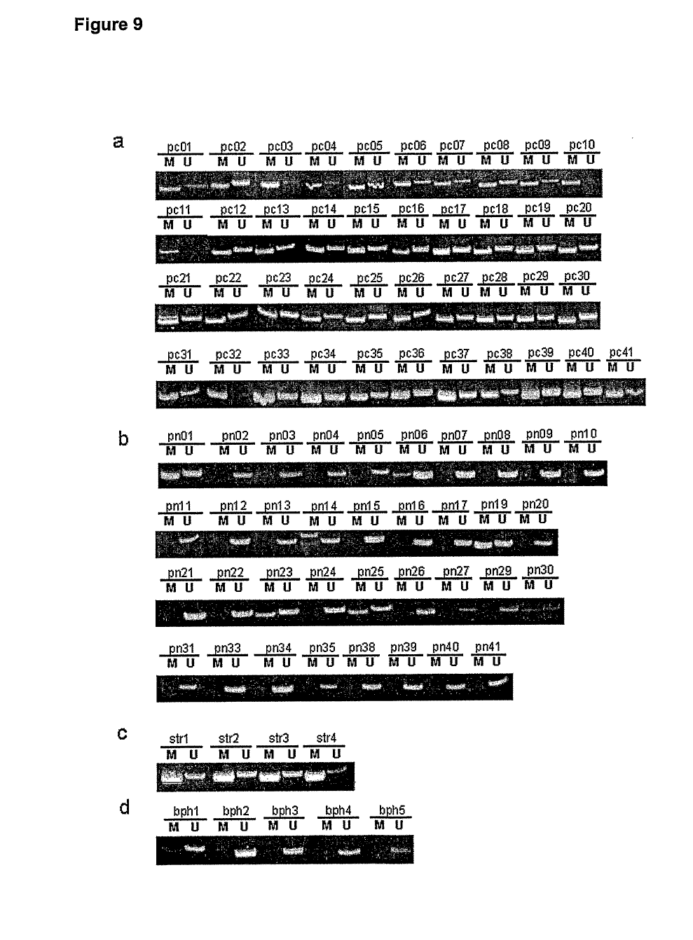

FIG. 9

14-3-3σ-specific MSP-analysis of in vivo CpG-methylation in prostatic tissue after laser-microdissection. Different neoplastic and non-neoplastic cell types were isolated from 5 μm sections of paraffin-embedded prostate derived from 41 different PCa and 9 BPH patients using laser-pressure catapulting.

(a) MSP analysis of 41 tumor cell samples and (b) 38 samples of corresponding normal prostate epithelial cell from the indicated patients. (c) MSP analysis of 4 stromal and (d) 5 BPH cell samples collected from 9 BPH patients.

Archival formalin-fixed, paraffin-embedded samples of primary prostate carcinoma (Gleason Sum 5-10) or cancer free samples of prostate (BPH) were obtained from the Institute of Pathology, Ludwig-Maximilians University, Munich. The specimens were de-paraffinized in xylene and briefly stained with hematoxylin and eosin. Microdissection and laser-pressure catapulting was performed using a MicroBeam system (P.A.L.M., Bernried, Germany). A covered section was used as a reference slide. Material obtained from 2-3 parallel sections (˜10³ cells) was pooled and genomic DNA isolated by the proteinase K/SDS method. MSP analysis was performed as described previously²⁹.

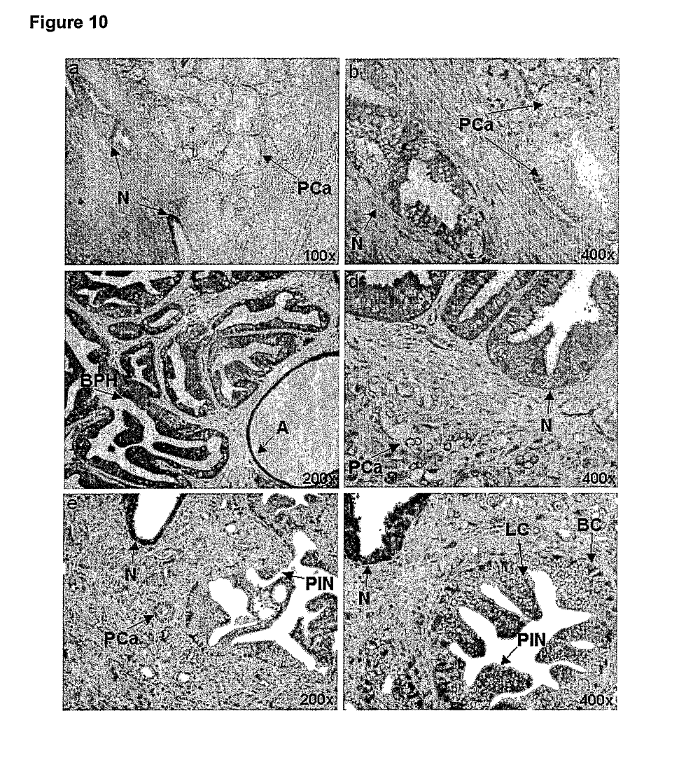

FIG. 10

Analysis of 14-3-3σ protein expression in primary PCa, PIN, BPH, and normal prostate epithelium. Depicted are immunohistochemical detections of 14-3-3σ protein (red color) in 3 μm sections of paraffin-embedded prostate tissue obtained from PCa and BPH patients. (A: atrophic prostate epithelial cells; BPH: benign prostate hyperplasia; N: non-neoplastic prostate epithelial cells; PCa: prostate cancer cells; PIN: prostatic intra-epithelial neoplasia). (a,b,d) sections containing normal prostate glands and PCa; (c) section with BPH and an atrophic gland; (e,f) section with PIN, PCa and normal prostate glands. The respective magnifications are indicated in the frames (x fold). The specificity and use of the affinity-purified rabbit polyclonal anti-serum specific for 14-3-3σ on paraffin-embedded sections was described previously²⁹. A 1:200 dilution of 14-3-3σ anti-serum was used in combination with the APAAP detection system (DAKO, Copenhagen, Denmark). After counterstaining with hematoxylin, the images were acquired using an Axiovert 200M microscope (Carl Zeiss Co., Oberkochen, Germany) coupled to a DXC-390P CCD camera (Sony, Tokyo, Japan) and PALM-Robo V2.1.1 software (P.A.L.M.).

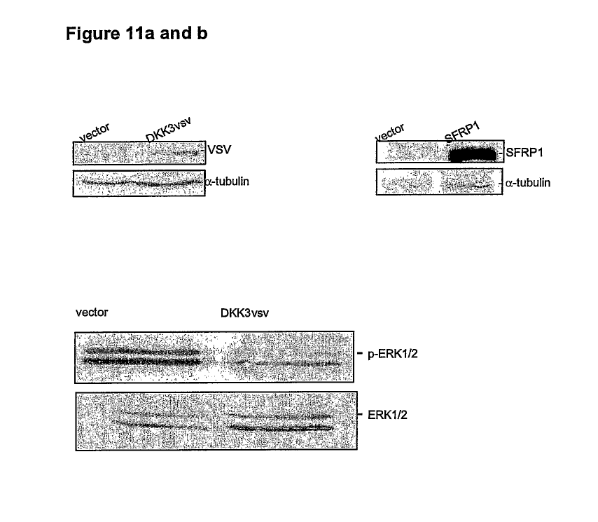

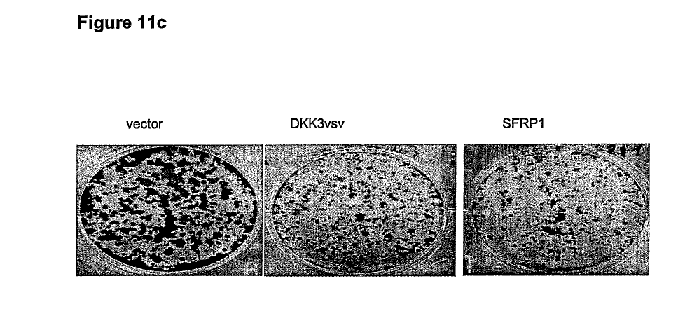

FIGS. 11a and 11b

Effects of ectopic DKK3 and SFRP1 expression in PCa cell lines.

a. Ectopic expression of DKK3 and SFRP1 in prostate carcinoma cells. PC3 cells were infected with bicistronic retroviruses encoding EGFP2, DKK3+EGFP2 or SFRP1+EGFP2; GFP-positive cells were isolated by flow cytometry and total protein lysates were subjected to Western blot analysis with anti-VSV or anti-SFRP1 antibodies. Equal loading was confirmed by detection of ±-tubulin.

b. Inhibition of ERK1/2 phosphorylation in PC3 cells ectopically expressing DKK3. Total protein lysates were analyzed using antibodies specific for ERK1 and ERK2 phosphorylated at the Thr202 and Tyr204 residues or anti-ERK1/2 antibodies.

c. Inhibition of cellular proliferation by DKK3 and SFRP1. Equal number of PC3 cells transduced with the indicated constructs were plated at low density in 6-well plates, cultured for 10 days and colonies were stained with crystal violet. The analysis was performed in duplicates yielding identical results (data not shown).

### EXAMPLE

### 1. Methods

### 1.1. Cell Culture and Tissue Samples

The PCa cell lines Du-145, LNCaP, PC3, PPC1 and TSU-Prl were cultured in RPMI-1640 supplemented with 10% fetal bovin serum (FBS) and antibiotics (Invitrogen). The PCa cell line LAPC-4 was kept in RPMI-1640 in the presence of 20% FBS. Human benign prostate hyperplasia cells immortalized with SV40 large T-antigen (BPH1) were obtained from the German Collection of Microorganisms and Cell Cultures and passaged in RPMI-1640 medium supplemented with 20% FBS, 20 ng/ml testosteron, 50 μg/ml transferrin, 50 ng/ml sodium selenite, 50 μg/ml insulin and a mixture of trace elements (Invitrogen). Human primary prostate epithelial cells (PrECs) from an 18-year old donor (Clonetics) were cultured in PrEGM according to the supplier's instructions on Collagen Type I coated dishes (BioCoat, BD Falcon). LNCaP and PC3 cells were seeded at low density 24 hours before de-methylation. Archival formalin fixed, paraffin-embedded samples of primary prostate carcinoma (Gleason Sum 5-10) and cancer free samples of prostate were obtained from the Institute of Pathology, Ludwig-Maximilians University, Munich.

### 1.2. Laser-Assisted Tissue Microdissection

Archival specimens of primary PCa and tumor free prostate tissue were deparaffinized in xylene and briefly stained with hematoxylin and eosin. One section was covered and used as a reference slide. Microdissection and laser-pressure catapulting was performed using a MicroBeam system (P.A.L.M.). Material obtained from 2-3 parallel sections (˜10³ cells) was pooled and genomic DNA was isolated by the proteinase K/SDS method (see below). Before bisulfite-treatment, 1 μg of herring sperm carrier DNA (Promega) was added to each sample of microdissected DNA.

### 1.3. Microarray Analysis

Total RNA was isolated from cell lines using the RNAgent kit (Promega) and assessed photometrically and by agarose gel electrophoresis. Oligonucleotide microarray analyses were performed on the GeneChip Human Genome U133A (list of genes is available at www.affymetrix.com) according to the manufacturer's protocol. In brief, 25 μg of total RNA were reverse transcribed using an anchored oligo-dT-T7 primer and the double-stranded cDNA synthesis kit (Invitrogen). Using the in vitro transcription RiboMax T7 kit (Promega), biotin-labelled cRNA was produced from a double-stranded cDNA template, fragmented, hybridized to U133A oligonucleotide arrays (15 μg of the RNA probe per chip) and analysed with a GeneChip® Scanner 3000. The U133B-array was not used since it repeatedly did not yield any positive results (data not shown). This may be due to the low abundance of mRNAs potentially detectable by this array. Genes up-regulated by combined 5Aza-2′dC plus TSA treatment versus TSA alone were identified by an algorithm provided by Affymetrix using the Microarray Suite 4.0 software.

### 1.4. RT-PCR Analysis

5 μg of the total RNA was reverse transcribed using an oligo-(dT)₁₈ primer and SuperScript™ double-stranded cDNA synthesis kit (Invitrogen) at 50° C. for 60 min in a total volume of 20 μl. cDNA was diluted two fold, first tested on the LightCycler with EF1α-specific primers (Table 2) using FastStart-DNA Master SYBR Green 1 kit (Roche Diagnostics), then diluted to equal concentrations and used for PCR. Primer sequences and annealing temperatures are given in Table 2. 2 units Platinum Taq polymerase (Invitrogen) were used per 20 μl reaction with 2 μl cDNA. The primer sequences and PCR cycle numbers for each analysed gene are provided in Table 2 below The total reaction was analysed by agarose gel electrophoresis.

### Table 2

Primers used for bisulfite-sequencing (a) (SEQ ID NO:1-196), MSP (b) (SEQ ID NO:197-286) and RT-PCR (c) (SEQ ID NO:287-312).

Sequence information of primers used for the amplification of bisulfite converted genomic DNA for the indicated gene is summarized. Non-converted sequence extracted from the NCBI human genome draft sequence is shown for all sequencing and MSP primers designed in this study for the unambiguous localization of amplified products and relative position of CpG-dinucleotides within them. Sequence modifications caused by the bisulfite-conversion are shown in colour; R=A or G, Y=C or T bases. For unreferenced RT-PCR primers, accession number of the source sequence is indicated.

TABLE 2

| | | | | |
|---|---|---|---|---|
| Gene | forward sequence 5′->3′ | reverse sequence 5′->3′ | Size | Ta |

Oligonucleotides used for bisulfite PCR

| | | | | | |
|---|---|---|---|---|---|
| APAF1 | non converted | ATAGTTCCCCTAGGAGAGGTGGG | CAGCCTGACCCCACAGTCCC | 682 | 60° |
| | sequence | ATAGTTTTTTTAGGAGAGGTGGG | CAACCTAACCCCACAATCCC | | |
| APPD | non converted | GCTGAGGGTGACTGTGCTGTGAGGC | CCCAGGCTCCCTGCAGGTTCACCCC | 665 | 60° |
| | sequence | GTTGAGGGTGATTGTGTTGTGAGGT | CCCAAACTCCCTACAAATTCACCCC | | |
| BIN1 | non converted | GGTGAGCCCCTGGAAAAGGAGGGGG | CCCTTTACTGCCCATCTCTGCCATC | 633 | 60° |
| | sequence | GGTGAGTTTTTGGAAAAGGAGGGGG | CCCTTTACTACCCATCTCTACCATC | | |
| BRCA2 | non converted | TGGCCTGGGACTCTTAAGGGTCAG | TTGGCAGAGACAAAAGGGCAAGAAGCC | 517 | 62° |
| | sequence | TGGTTTGGGATTTTTAAGGGTTAG | TTAACAAAAACAAAAAAACAAAAAACC | | |
| BTG1 | non converted | CCCCTAGGGTGGAACAGAAATGGCT | ATCTCAATAGCTGCATTTCCAGCTC | 771 | 65° |
| upstream | sequence | TTTTTAGGGTGGAATAGAAATGGTT | ATCTCAATAACTACATTTCCAACTC | | |
| | non converted | GAGCTGGAAATGCAGCTATTGAGAT | CCACGGCTCCTTTGTCCCCAAATCC | 771 | 65° |
| downstream | sequence | GAGTTGGAAATGTAGTTATTGAGAT | CCACRACTCCTTTATCCCCAAATCC | | |
| BTG3 | non converted | GGTCTAGAGAGCTGGGTCTAGAACT | CCCCTGCCCCTCCCCTGTCCCC | 785 | 60° |
| | sequence | GGTTTAGAGAGTTGGGTTTAGAATT | CCCCTACCCCTCCCCTATCCCC | | |
| CASP7 | non converted | GGTACTTCCTTCAAAGCTGAGGGAG | CAACCAGGCTCCCCTAGACCAC | 883 | 65° |
| | sequence | GGTATTTTTTTTAAAGTTGAGGGAG | CAACCAAACTCCCCTAAACCAC | | |
| CITED2 | non converted | GAGGCACAAGGGCACTCTGGAGGG | CAGCACATAGAGGGGACCTTCCTGGC | 630 | 60° |
| | sequence | GAGGTATAAGGGTATTTTGGAGGG | CAACACATAAAAAAAACCTTCCTAAC | | |
| CTGF | non converted | GTGTAGGACTCCATTCAGCTCATTGG | CCGAAGTGACAGAATAGGCCCTTGTGC | 648 | 60° |
| | sequence | GTGTAGGATTTTATTTAGTTTATTGG | CCRAAATAACAAAATAAACCCTTATAC | | |
| CUTL2 | non converted | GGAGCTGGGGGTAGACAGGTGCAAG | CAATGGCTGCACTCAATATCCGGGCTGGAC | 548 | 62° |
| | sequence | GGAGTTGGGGGTAGATAGGTGTAAG | CAATAACTACACTCAATATCCRAACTAAAC | | |
| CYLD | non converted | GAGGAAGGTCTGTCACAGGGAGG | CGCCATTAACAAGGCCAGAACCCC | 806 | 65° |
| | sequence | GAGGAAGGTTTGTTATAGGGAGG | CRCCATTAACAAAACCAAAACCCC | | |
| DDB2 | non converted | GAAAGGCACTAGCTCTCTACAAAGC | GGGCTTGTTCAAACCAGCTTGGAGC | 665 | 57° |
| | sequence | GAAAGGTATTAGTTTTTTATAAAGT | AAACTTATTCAAACCAACTTAAAAC | | |
| DKC1 | non converted | GAAAGCAAAGAAAGAGGTACTGTTTA | CTGGCAGCAGCACAGACACTGCCAC | 764 | 65° |
| | sequence | GAAAGTAAAGAAAGAGGTATTGTTTA | CTAACAACAACACAAACACTACCAC | | |
| DKK1 | non converted | AGGCAAGGGCACCCAAGTTCCCAGAGT | CTGACTGCAGAGCCTGGGTGCCCC | 631 | 64° |
| | sequence | AGGTAAGGGTATTTAAGTTTTTAGAGT | CTAACTACAAAACCTAAATACCCC | | |
| DKK3 | non converted | GCAGGCAGTGAAGGAGATGGCTG | TACCTGGGGTGGACCAAGCACAGGTCA | 505 | 64° |
| upstream (a) | sequence | GTAGGTAGTGAAGGAGATGGTTG | TACCTAAAATAAACCAAACACAAATCA | | |
| | non converted | TGAGGAGCAGAGCTCAGCTTGTGC | CATCTCATTGAGGGTGGCCTCCTCC | 436 | 64° |
| downstream(b) | sequence | TGAGGAGTAGAGTTTAGTTTGTGT | CATCTCATTAAAAATAACCTCCTCC | | |
| DLC1 | non converted | GCTCAAGGCACACTAGGGTCCAGGC | GCTTCTTTCTGCACATCAAGCAC | 759 | 60° |
| | sequence | GTTTAAGGTATATTAGGGTTTAGGT | ACTTCTTTCTACACATCAAACAC | | |
| DUSP1 | non converted | GGAGGGAGAGAGGGAGGAG | CCCACTTCCATGACCATGGC | 666 | 61° |
| | sequence | GGAGGGAGAGAGGGAGGAG | CCCACTTCCATAACCATAAC | | |
| GADD45A | non converted | AAGACATGAAAAGATAATAAGAAAAAAGTG | CTTCCTCCCCTGCAAGCCTTCCACAGCCC | 813 | 65° |
| | sequence | AAGATATGAAAAGATAATAAGAAAAAAGTG | CTTCCTCCCCTACAAACCTTCCACAACCC | | |
| GAS2L1 | non converted | TCCCCAGGAGACCAAAGAGGTTGGA | TGGGGCTCTGGGCCCAGAGAGGGTG | 549 | 57° |
| | sequence | TTTTTAGGAGATTAAAGAGGTTGGA | TAAAACTCTAAACCCAAAAAAAATA | | |
| GPX3 | non converted | GCCCCCTTGCCCTGGCTGTAATGGAGAC | CTGGGAACTTGCACAGCCCACCCAGAC | 860 | 65° |
| | sequence | GTTTTTTTGTTTTGGTTGTAATGGAGAT | CTAAAAACTTACACAACCCACCCAAAC | | |
| GSTM1 | non converted | GTTAGGATCTGGCTGGTGTCTCAAG | CCAGGGAAGCCCCCAGTTTACACTGC | 596 | 60° |
| | sequence | GTTAGGATTTGGTTGGTGTTTTAAG | CCAAAAAAACCCCCAATTTACACTAC | | |
| HPGD | non converted | AGCCAGAGGCTGAGGGGAGGCTTTG | TGAGGTGTGCTCACAGCCTCAGCTTC | 514 | 66° |
| | sequence | AGTTAGAGGTTGAGGGGAGGTTTTG | TAAAATATACTCACAACCTCAACTTC | | |
| HUS1 | non converted | TTAAAATAACACTTGAAATAGGTGTCA | CCTTCATCCCCACAAGTGCCCTCC | 655 | 60° |
| | sequence | TTAAAATAATATTTGAAATAGGTGTTA | CCTTCATCCCCACAAATACCCTCC | | |
| ICAM1 | non converted | GAGGGGCATCCCTCAGTGGAGGGAGC | CTACCTAAGCATGCATGACCTGACCC | 725 | 64° |
| | sequence | GAGGGGTATTTTTTAGTGGAGGGAGT | CTACCTAAACATACATAACCTAACCC | | |
| ID3 | non converted | AGTCTGGAGGTCAGACGAACAGCAAATTGG | CATCCTTGCCTGGGTGTTCAGCCCTGTC | 916 | 65° |
| | sequence | AGTTTGGAGGTTAGAYGAATAGTAAATTGG | CATCCTTACCTAAATATTCAACCCTATC | | |
| IRF1 | non converted | GAAAAGATGGCCCCAGGAGCCAG | CCCCCCACTTCCTGGTGCCC | 666 | 61° |
| | sequence | GAAAAGATGGTTTTAGGAGTTAG | CCCCCCACTTCCTAATACCC | | |
| IRF7 | non converted | TGTCCCCTGGGCTGTAGTGGAGTGGC | CAGGTGTGGACTGAGGGCTTGTAGCCACC | 665 | 58° |
| | sequence | TGTTTTTTGGGTTGTAGTGGAGTGGT | CAAATATAAACTAAAAACTTATAACCACC | | |
| JUNB | non converted | TGAAACCCCTCACTCATGTGCCTGGG | AGGGCTGTTCCATTTTAGTGCACATC | 590 | 60° |
| | sequence | TGAAATTTTTTATTTATGTGTTTGGG | AAAACTATTCCATTTTAATACACATC | | |
| MRE11 | non converted | GGAGGGAGAGGGGATCCAGCTC | CCAGGACCCCTCCCCTGCCCACTC | 714 | 64° |
| | sequence | GGAGGGAGAGGGGATTTAGTTT | CCAAAACCCCTCCCCTACCCACTC | | |
| NGFR | non converted | GATGGGTAAGAGAGTGAACCCTGTGG | CTCACCCCCAGAAGCAGCAACAGCAGC | 529 | 64° |
| | sequence | GATGGGTAAGAGAGTGAATTTTGTGG | CTCACCCCCAAAAACAACAACAACAAC | | |
| p19 | non converted | TTGCCACACTCTGACCAATCAGGAG | ACCAGAGAGGAGCTCTGGGGTCTC | 792 | 58° |
| (CDKN2D) | sequence | TTGTTATATTTTGATTAATTAGGAG | ACCAAAAAAAAACTCTAAAATCTC | | |
| p21 | non converted | GGGACCGGCTGGCCTGCTGGAACT | CTTCCTGGGCCCCTCCAGGGACAC | 697 | 65° |
| (CDKN1A) | sequence | GGGATTGGTTGGTTTGTTGGAATT | CTTCCTAAACCCCTCCAAAAACAC | | |
| p57 | non converted | GGCTGGGCGTTCCACAGGCCAAGTG | CCGGGACACTAGGCAGCTGCTCC | 718 | 63° |
| (CDKN1C) | sequence | GGTTGGGYGTTTTATAGGTTAAGTG | CCRAAACACTAAACAACTACTCC | | |
| p130 | non converted | TTCACCCCTGGTGAAACTAGGGGAG | CAGAGGAAGTCCCCACCCCTCTC | 621 | 65° |
| (RBL2) | sequence | TTTATTTTTGGTGAAATTAGGGGAG | CAAAAAAAATCCCCACCCCTCTC | | |
| PMS2 | non converted | CATAAAAGTCTGAGTGAGTCCCTGGC | GCCATGTTCCCCCCATTTCCAGGG | 749 | 64° |
| | sequence | TATAAAAGTTTGAGTGAGTTTTTGGT | ACCATATTCCCCCCATTTCCAAAA | | |
| PTGER4 | non converted | AGAAAAGTTTGTACAGAGGGTGGAA | TCAGTGAAGAATGGTGCTGGATTTC | 355 | 53° |
| | sequence | AGAAAAGTTTGTATAGAGGGTGGAA | TCAATAAAAAATAATACTAAATTTC | | |
| PTGS2 b | non converted | TCAGTCTTATAAAAAGGAAGGTTCT | TGCTTGTGGGAAAGCTGGAATATCC | 413 | 52° |
| | sequence | TTAGTTTTATAAAAAGGAAGGTTTT | TACTTATAAAAAAACTAAAATATCC | | |
| PTGS2 a | non converted | ATCAGACAGGAGAGTGGGGAC | AGCTCCACAGCCAGACGCCCTCAGA | 331 | 55° |
| | sequence | ATTAGATAGGAGAGTGGGGAT | AACTCCACAACCAAACRCCCTCAAA | | |
| RIS1 | non converted | GGGCCCTGGAGCCTCCCTCTGAGAA | AGCAGTAGGTCGCACTGGAAGCCCC | 850 | 64° |
| | sequence | GGGTTTTGGAGTTTTTTTTTGAGAA | AACAATAAATCRCACTAAAAACCCC | | |
| SGK | non converted | TGGGGGCTTGGCTCACTTCCCCAGA | GGACTTTCAAAAAATTTCCACTTTG | 855 | 60° |
| | sequence | TGGGGGTTTGGTTTATTTTTTTAGA | AAACTTTCAAAAAATTTCCACTTTA | | |
| SMARCA1 | non converted | AAAGAGCAGATTTAAGGGAAAGAGG | CCACCACACACACACCCCCTTCCT | 659 | 64° |
| | sequence | AAAGAGTAGATTTAAGGGAAAGAGG | CCACCACACACACACCCCCTTCCT | | |
| SNK | non converted | TTTAGTACTAGTAACTGTCAAAGGC | GGCGGCTGGCTGGTAGGTGATAGT | 1084 | 52° |
| | sequence | TTTAGTATTAGTAATTGTTAAAGGT | AACAACTAACTAATAAATAATAAT | | |
| SQSTM1 | non converted | GGAAGGGGAGAGTAGTGAAGGGG | CCTTGGTCACCACTCCAGTCACCA | 593 | 65° |
| | sequence | GGAAGGGGAGAGTAGTGAAGGGG | CCTTAATCACCACTCCAGTCACCA | | |
| STK38L | non converted | CCACTCTCAAGAGAGGCCTGAACAG | ACCTAAAATCTCCTCCTGCTCCTGC | 493 | 60° |
| | sequence | TTATTTTTAAGAGAGGTTTGAATAG | ACCTAAAATCTCCTCCTACTCCTAC | | |
| TNFRSF10B | non converted | CCCTGGGAAGGGGAGAAGATCAAGA | CCCTCTCTCCCTGCCCTCTCCAGGC | 744 | 65° |
| | sequence | TTTTGGGAAGGGGAGAAGATTAAGA | CCCTCTCTCCCTACCCTCTCCAAAC | | |
| XPC | non converted | GGAAAAAGCAGCCTAGTACAAGAAGCT | CTCTTGGCCTTGGATTTCTGGCTGC | 445 | 62° |
| | sequence | GGAAAAAGTAGTTTAGTATAAGAAGTT | CTCTTAACCTTAAATTTCTAACTAC | | |
| ZFP36 | non converted | AGTCTCCAGCTTTGAAAACTGGGCAGG | CCTCCAAATCACCAAGCTGGTCTGAGC | 536 | 65° |
| | sequence | AGTTTTTAGTTTTGAAAATTGGGTAGG | CCTCCAAATCACCAAACTAATCTAAAC | | |

Oligonucleotides used for MSP analysis

| | | | | | |
|---|---|---|---|---|---|
| SFRP1 | MSP (M) | TGTAGTTTTCGGAGTTAGTGTCGCGC | CCTACGATCGAAAACGACGCGAACG | 127 | 65° |
| | MSP (U) | GTTTTGTAGTTTTTGGAGTTAGTGTTGTGT | CTCAACCTACAATCAAAAACAACACAAACA | 136 | 65° |
| APOD | MSP (M) | CACACGCGCGAAAACAATAT | TATGTATGTTACGTTCGTCG | | 59° |
| | MSP (U) | CACACAAAAACAATATCTCATTTCT | TTTTTTATGTATGTTATGTTTGTTG | | 55° |
| RASSF1A | MSP (M) | GGGTTTTGCGAGAGCGCG | GCTAACAAACGCGAACCG | 169 | 64° |
| | MSP (U) | GGGGTTTTGTGAGAGTGTGTTTAG | TAAACACTAACAAACACAAACCAAAC | 175 | 62° |
| 14-3-3sigma | MSP (M) | TGGTAGTTTTTATGAAAGGCGTC | CCTCTAACCGCCCACCACG | 105 | 65° |
| | MSP (U) | ATGGTAGTTTTTATGAAAGGTGTT | CCCTCTAACCACCCACCACA | 107 | 65° |
| p16 | MSP (M) | TTATTAGAGGGTGGGGCGGATCGC | GACCCCGAACCGCGACCGTAA | 150 | 65° |
| (CDKN2A) | MSP (U) | TTATTAGAGGGTGGGGTGGATTGT | CAACCCCAAACCACAACCATAA | 151 | 60° |
| GSTP1 | MSP (M) | *GGTTTTTTCGGTTAGTTGCGCGGCG | CCAACGAAAACCTCGCGACCTCCG | 206 | 60° |
| | MSP (U) | AAAGAGGGAAAGGTTTTTTTGGTTAGTTGTGTGGTG | AAACTCCAACAAAAACCTCACAACCTCCA | 222 | 64° |
| RIZ1 | MSP (M) | GTGGTGGTTATTGGGCGACGGC | GCTATTTCGCCGACCCCGACG | 127 | 68° |
| | MSP (U) | TGGTGGTTATTGGGTGATGGT | ACTATTTCACCAACCCCAAGA | 126 | 60° |
| THBS1 | MSP (M) | TGCGAGCGTTTTTTTAAATGC | TAAACTCGCAAACCAACTCG | 74 | 62° |
| | MSP (U) | GTTTGGTTGTTGTTTATTGGTTG | CCTAAACTCACAAACCAACTCA | 115 | 62° |
| TFPI2 | MSP (M) | CACAATTTACAACGCGAAAACGACGAA | TTTTGTTTTAGGCGGTTCGGGTGTTC | 135 | 65° |
| | MSP (U) | CTTACACAATTTACAACACAAAAACAACAAA | GTTTTTTGTTTTAGGTGGTTTGGGTGTTT | 142 | 65° |
| PTGS2 | non converted | TAAAAAACCCTGCCCCCACCGGGCTTACG | AGTCGTATGACAATTGGTCGCTAACCGAG | | |
| | MSP (M) | AAAATTTTGTTTTTATCGGGTTTAC | CGTATAACAATTAATCGCTAACCG | 165 | 60° |
| | MSP (U) | AAAAAATTTTGTTTTTATTGGGTTTAT | AATCATATAACAATTAATCACTAACCA | 170 | 60° |
| GPX3 | non converted | TGGGGAGCTGAGGGCAAGTCGCGCCCGCC | GCTGCCTGATCCCTGGCCACCGTCCGT | | |
| | MSP (M) | GGAGTTGAGGGTAAGTCGCGTTC | ACCTAATCCCTAACCACCGTCCG | 156 | 65° |
| | MSP (U) | GGGGAGTTGAGGGTAAGTTGTGTTT | CTACCTAATCCCTAACCACCATCCA | 160 | 65° |
| DDB2 | non converted | GACCCGCAGAGGCCTGGCAGCGCCGCGTT | TACAAGCGTACGCCACCACGCCCGGC | | |
| | MSP (M) | GTAGAGGTTTGGTAGCGTCGC | GTACGCCACCACGCCCG | 175 | t/d |
| | MSP (U) | TTTGTAGAGGTTTGGTAGTGTTGT | AACATACACCACCACACCCA | 181 | t/d |
| GSTM1 | non converted | TAGGGAAGCTGGCGAGGCCGAGCCCCGCC | CTCGGCCCGCCACAACCCGAAAGGCGCC | | |
| | MSP (M) | GAAGTTGGCGAGGTCGAGTTTC | ACCCGCCACAACCCGAAAAACG | 157 | t/d |
| | MSP (U) | GGGAAGTTGGTGAGGTTGAGTTTT | CAACCCACCACAACCCAAAAAACA | 161 | t/d |
| DKK3 | non converted | GTGAGGTGGCCGGCGCCCCGGCTGGC | CCCACCAGGCTCCCTCTCCGCCCCCGCC | | |
| upstream | MSP (M) | AGGTGGTCGGCGTTTCGGTTGG | CCAAACTCCCTCTCCGCCCCCG | 109 | t/d |
| (a) | MSP (U) | TGAGGTGGTTGGTGTTTTGGTTGG | CACCAAACTCCTCCTCCACCCCCA | 113 | t/d |
| downstream | non converted | GCCAGGGGCGGGCGGCGGGGCGG | CGCTGCATCTCCGCTCTGCGCCCGC | | |
| (b) | MSP (M) | GGGGCGGGCGGCGGGGC | ACATCTCCGCTCTACGCCCG | 120 | t/d |
| | MSP (U) | TTAGGGGTGGGTGGTGGGGT | CTACATCTCCACTCTACACCCA | 125 | t/d |
| RIS1 | non converted | CCCGTCCGCGGCGGGGCCGCGGACGCGCC | GGGGCCGCCGAGGCCAGCAGCC | | |
| | MSP (M) | TCGCGGCGGGGTCGCGGACGCG | AAAACCRCCRAAACCAACAACC | 113 | t/d |
| | MSP (U) | GTTTGTGGTGGGGTTGTGGATGTG | AAAACCRCCRAAACCAACAACC | 115 | t/d |
| p57 | non converted | GCTGCCCGCGTTTGCGCAGCCCCGGG | GGGAAGGTCCCACGGGCGACAAGACGCT | | |
| (CDKN1C) | MSP (M) | TTGTTYGYGTTTGCGTAGTTTC | AAATCCCACRAACGACAAAACG | 88 | 62° |
| | MSP (U) | GTTGTTYGYGTTTGTGTAGTTTT | AAAAATCCCACRAACAACAAAACA | 91 | 62° |
| HPGD | non converted | GGGGGCATAAAAGCCGCGGCCGCGCGG | GCAGAGAAATTTCCGCGGCTGGGCGCCGGG | | |
| | MSP (M) | GTATAAAAGTCGCGGTCGCGC | AAATTTCCGCGACTAAACGCCG | 189 | 65° |
| | MSP (U) | GGGTATAAAAGTTGTGGTTGTGT | AAAAAAATTTCCACAACTAAACACCA | 195 | 65° |

t/d: touch down PCR with 2 cycles at 68° C., 2 cycles at 66° C., X cycles at 65° C.
*: error in the reference: one extra T in the M forward primer sequence

| | | | | | |
|---|---|---|---|---|---|
| | | | | | Cy- |
| Gene | size | forward sequence 5′->3′ | reverse sequence 5′->3′ | Ta | cles |

Oligonucleotides used for RT-PCR

| | | | | | |
|---|---|---|---|---|---|
| 14-3-3σ | 99 | CCAGGCTACTTCTCCCCTC | CTGTCCAGTTCTCAGCCACA | 60° | 31 |
| APOD | 498 | AAAAGCTCCAGGTCCCTTC | AGGGTTTCTTGCCAAGATCC | 58° | 25 |
| DKK3 | 179 | AAGGCAGAAGGAGCCACGAGTGC | GGCCATTTTGGTGCAGTGACCCCA | 60° | 34 |
| GSTM1 | 191 | ACTTTCCCAATCTGCCCTAC | TTCTGGATTGTAGCAGATCA | 55° | 32 |
| GSTP1 | 242 | TCACTCAAAGCCTCCTGCCTAT | CAGTGCCTTCACATAGTCATCC | 60° | 33 |
| GPX3 | 134 | CTTCCTACCCTCAAGTATGTCCG | GAGGTGGGAGGACAGGAGTTCTT | 65° | 33 |
| EF1A | 192 | CACACGGCTCACATTGCAT | CACGAACAGCAAAGCGACC | 60° | 18 |
| HPGD | 149 | CTCTGCAAATTAATTTGGTTTCTGT | ACAGAAACCAAATTAATTTGCAGAG | 64° | 26 |
| RASSF1A | 239 | TGGTGCGACCTCTGTGGCGACTT | TCCTGCAAGGAGGGTGGCTTC | 60° | 37 |
| p57 | 341 | CTGACCAGCTGCACTCGGGGATTTC | GCCGCCGGTTGCTGCTACATGA | 60° | 31 |
| p16 | 139 | AGCCTTCGGCTGACTGGCTGG | CTGCCCATCATCATGACCTGGA | 60° | 40 |
| PTGS2 | 193 | TAAACAGACATTTATTTCCAGAC | GAAAGAAATAGTCAATATGCTTG | 60° | 33 |
| SFRP1 | 532 | TTGTAGTTATCTTAGAAGATAGCATGG | ACGGGAATTACTATTAACATAAGCG | 65° | 31 |

### 1.5. Bisulfite Treatment of Genomic DNA

Genomic DNA was isolated by overnight incubation in a solution containing 100 μg/ml proteinase K (Sigma) and 0.1% SDS (Sigma) at 55° C. with subsequent phenol/chloroform extraction and isopropanol precipitation. 2 μg DNA were denatured in 0.2 M NaOH for 10 min at 37° C. in 50 μl total volume. After addition of 30 μl of 10 mM hydroquinone (Sigma) and 520 μl of 3.5 M sodium bisulfite pH 5.0 (Sigma), the mixture was incubated for 16 hours at 50° C. After column-purification (Qiagen), DNA was incubated in 0.3 M NaOH for 5 min at RT. Converted DNA was ethanol precipitated and dissolved in 40 μl TE buffer. 2 μl and 5 μl were used for a single MSP or bisulfite-PCR reaction, respectively.

### 1.6. Genomic Bisulfite Sequencing

Converted gDNA was used as a template to amplify regions of interest (400-1000 bp fragments with a high CpG-content around the transcription start site) by PCR using gene specific primers (listed in Table 2). After 5 min incubation at 95° C., 39-41 cycles were performed: 20 s at 95° C., 30 s at annealing temperature, 60-90 s at 72° C. using 5 units of Platinum Taq polymerase (Invitrogen) per 100 μl reaction. PCR-products were isolated by gel purification and subcloned in a TOPO-TA vector (Invitrogen). At least 6 individual clones for each gene were sequenced in both directions using M13 primers and BigDye terminator, and analysed on a 3700 capillary sequencer (Applera).

### 1.7. MSP-Analysis

MSP was performed in a total volume of 20 μl using 3 units Platinum Taq per reaction and gene specific primer sets (Table 2), discriminating between methylated and unmethylated DNA. After 5 min denaturation at 95° C., 40 PCR-cycles were performed for gDNA obtained from cell lines and 45 for micro-dissected gDNA. Amplified fragments were separated by 8% polyacrylamide gel electrophoresis and visualized by ethidium bromide staining.

### 1.8. Immunohistochemical Staining

The use of the affinity-purified rabbit polyclonal 14-3-3σ specific anti-serum on paraffin-embedded sections was described previously²⁹. Goat polyclonal antibodies against SFRP1 (c-19) were from Santa Cruz Biotechnology. 6 μm sections were deparaffinized in xylene, re-hydrated in decreasing ethanol series and subjected to antigen retrieval procedure: boiled in a microwave oven for 15 min in citrate pH 6.0 buffer for 14-3-3τσ IHC and 30 min in ProTaqs IV buffer (Biocyc) for SFRP1 IHC. 1:200 dilution of 14-3-3σ anti-serum was used in combination with the APAAP detection system (DAKO). Anti-SFRP1 antibodies diluted 1:25 were used with Vectastain Elite ABC kit (Vector Laboratories). After counterstaining with hematoxylin, the images were acquired on an Axiovert 200M microscope (Zeiss) coupled to a DXC-390P CCD camera (Sony) using a PALMRobo V2.1.1 software (P.A.L.M.).

1.9 Western Blot Analysis. Western blot analysis was performed as described previously (15). Antibodies used were directed against ±-tubulin (Santa Cruz), VSV (Sigma), phospho ERK1/2 and ERK1/2 (Cell Signaling Technology, Frankfurt, Germany). Secondary HRP-conjugated anti-mouse and anti-rabbit antibodies (Promega) were used at the dilution 1:5000.

1.10 Transfection and Luciferase Reporter Assay. The plasmids pGL3-OT, pGL3-OF and pcDNA3.1-His-WNT1 have been described previously (16). LNCaP, PC3, Du-145 and HCT116 cells were plated at medium density in 12-well plates 24 hours before transfection. Three constructs were co-transfected using Lipofectamin2000 reagent (Invitrogen): (i) 0.5 μg of pGL3-OT or pGL3-OF; (ii) 0.5 μg of pcDNA3.1-His-WNT1 or pcDNA3.1-His-A (Invitrogen), (iii) 50 ng of pCMVβ-gal (Promega). Transfections were performed in triplicates. After 36 hours cells were assayed for luciferase activity using a Luciferase Assay System kit (Promega) and for 1-galactosidase activity with a Galacto-Light kit (Tropix, Bedford, Mass.) on a MicroLumatPlus LB96V luminometer (EG&G Berthold, Bad Wildbad, Germany).

1.11 Generation and Analysis of Transgenic Cell Lines. For stable expression of DKK3 or SFRP1 the retroviral vector pLXSN (BD Clontech, Heidelberg, Germany), which had been modified by insertion of a IRES-EGFP fragment derived from the plasmid pIRES-EGFP2 (BD Clontech), was used. PC3 cells were retrovirally infected using pLXSN-IRES-EGFP2, pL-DKK3vsv-IRES-EGFP2 or pL-SFRP1-IRESEGFP2. 72 hours after infection GFP positive cells were sorted by FACS and expanded. For the assessment of colony formation cells were seeded at low density in 6-well plate (2000 cells per well) and grown for 10 days. Cells were fixed in 1% formaldehyde and stained with crystal violet. Apoptosis was assessed by propidium iodide staining and flow cytometry as described previously (15).

### 2. Results

In order to identify silenced tumor-suppressive genes involved in PCa formation we optimised the conditions for maximal re-expression of CpG-methylated genes in several PCa cell lines after a combined treatment with 5-Aza-2′deoxycytidine (5Aza-2′dC) and trichostatin A (TSA). The three PCa cell lines PC3, LNCaP and DU-145, which are derived from metastatic prostate carcinoma, were selected for further analysis since they showed the most effective de-methylation as detected by methylation-specific PCR (MSP²³) analysis (FIG. 1a). For subsequent micro-array analysis, RNA was isolated from these cell lines after exposure to 1 μM 5Aza-2′dC for 72 hours and 300 nM TSA for the last 24 hours or, as a control, to 300 nM TSA for 24 hours. RNA was converted to biotinylated cRNA and hybridized to oligonucleotide arrays representing ˜18,400 individual transcripts. In parallel, genomic DNA was isolated from all states, to confirm efficient de-methylation of CpG-dinucleotides (data not shown). Several hundred genes were found to be induced after treatment with 5Aza-2′dC plus TSA vs. TSA alone. Genes known to be imprinted (e.g. IGF2) or CpG-methylated in somatic tissues (e.g. MAGE) as well as target genes of the interferon pathway, which are unspecifically activated by 5Aza-2′dC treatment²⁴, were excluded from our analysis. Furthermore, a cut-off at 1.8 fold induction was chosen because GSTP1, a gene known to be hypermethylated in the majority of PCa¹⁵, was induced 1.87 fold after combined 5Aza-2′dC/TSA treatment of LNCaP cells. The induction of GSTP1 was confirmed by RT-PCR (FIG. 1a) and Northern blot analysis (FIG. 1b). Exemplary confirmations of re-expression detected by micro-array analyses were performed for 10 different genes by RT-PCR (FIG. 1a). In addition, expression of RASSF1A and p16, which are known to be induced after demethylation, was analysed by RT-PCR (FIG. 1a). Confirmation of re-induction after combined 5Aza-2′dC/TSA treatment of all tested genes indicated that the results of the micro-array analysis may be used to identify silenced genes. Genes found to be induced more than 1.8 fold by micro-array analysis were examined for potential, known tumor-suppressive functions (e.g. involvement in DNA-repair, induction of apoptosis, detoxification, differentiation, transcriptional regulation). Genes known to exhibit one of these functions were further examined for the presence of CpG-islands in their promoters. In total, 50 genes which met these criteria were selected for further analysis (listed in Table 3). Supporting the effectiveness of this approach, six genes previously shown to be down-regulated due to hyper-methylation in other types of tumors were among these genes: 14-3-3sigma, SFRP1, APOD, RIZ1, THBS1 and TFPI2^{21,22,25-27}.

### Methylation Pattern Analysis of 44 Candidate Promoters

For the remaining 44 candidate genes, the promoter regions were analysed by bisulfite-sequencing in order to determine the pattern of CpG-methylation in PCa and to allow the subsequent design of MSP-primers²³. Bisulfite treatment of genomic DNA leads to conversion of unmethylated cytosine to thymidine residues, whereas methylated CpG-residues are protected from de-amination. The promoter regions of 44 genes were analyzed by subcloning and sequencing of at least 6 independent clones of each promoter (see Table 2 for primer information). Extensive CpG-methylation was detected in the 5′ regions of DDB2, COX21PTGS2, GSTM1, HPGD, GPX3 and DKK3, whereas p57 and RIS1 displayed focal CpG-methylation (FIGS. 2a and 2b).

In order to determine whether these patterns can also be found in primary tumors, bisulfite-sequencing was performed on genomic DNA obtained after laser-microdissection of 2 primary, paraffin-embedded PCa-containing prostate sections (FIG. 2b). For the two genes analysed by bisulfite sequencing (GSTM1 and DKK3), the pattern of CpG-methylation was similar to the pattern found in cell lines and correlated with results obtained by MSP analysis (FIG. 2c). Unexpectedly, 35 of the 44 genes analysed by bisulfite sequencing did not show detectable CpG-methylation, although these genes were up-regulated after the combined 5Aza-2′dC and TSA treatment. Presumably, these genes were induced as a secondary consequence of the up-regulation of genes silenced by CpG-methylation or by the stress imposed by the 5Aza-2′dC/TSA treatment²⁸. These genes are also listed in Table 2 since they represent valuable information for investigators aiming to identify genes silenced in tumors using a similar approach. It is remotely possible that the CpG-dinucleotides responsible for the silencing of these genes were not in the region analysed by bisulfite sequencing in this study. However, as indicated in FIG. 2, the regions chosen for analysis cover several hundred base-pairs with a high CpG-content around the transcription start site, which are expected to be the main targets for CpG-methylation.

### MSP-Analysis in Prostate Cancer and Normal Cell Lines

Methylation-specific PCR (MSP) allows to efficiently assess the CpG-methylation status of promoters in a large number of samples and is therefore suited to determine the frequencies of CpG-methylation in larger cohorts of cancer tissue samples and cell lines. Based on the CpG-methylation pattern obtained by bisulfite-sequencing, MSP-primers were designed for 8 genes (indicated in FIG. 2a). For COX21PTGS2, the second primer was obtained from the literature (see Table 2) and for CUTL2, reliable MSP-conditions could not be established. For analysis of genes previously known to be methylated in other tumor types, the respective published MSP-primers were tested and used for MSP-analysis (see Table 2 for primer sequences). The CpG-methylation of 14 genes was examined by MSP in a panel of six prostate cancer cell lines, one cell line established from a benign prostate hyperplasia (BPH1) and, as a control, in primary prostate epithelial cells (PrECs; FIG. 3). This analysis revealed, that the genes initially identified in one of the cell lines PC3, LNCaP or Du-145 are also CpG-methylated in other PCa cell lines and occasionally in the BPH1 cell line. The results also confirmed that those genes were methylated at CpG-dinucleotides in those cell lines in which their re-expression was detected after treatment with 5Aza-2′dC and TSA (see column 4 in Table 3). The only exceptions were RIZ1, TFPI2 and THBS1, which did not show any CpG-methylation as determined by MSP-analysis in the cell lines used for the micro-array analysis, but in other PCa cell lines.

9 of the 14 genes analysed did not display CpG-methylation in primary prostate epithelial cells (PrECs). CpG-methylation at the CpG-sites interrogated by MSP-analysis of these 9 genes may be a specific feature of cancerous prostate epithelial cells. DDB2, GSTM1, APOD and RIS1 were at least partially methylated in PrECs (FIG. 3). However, in the case of DDB2 and GSTM1 the CpG-methylation appeared to be significantly elevated in most of the PCa-cell lines when compared to normal PrECs (FIG. 3), suggesting a PCa-specific increase in CpG-methylation of these genes and potential subsequent silencing.

In order to determine whether CpG methylation correlates with reduced or absent expression of the respective genes we analyzed the expression level of 9 genes which showed selective or preferential CpG methylation in PCa. RT-PCR analysis of cDNAs obtained from normal prostate epithelial cells derived from 4 healthy donors and 4 PCa cell lines revealed several distinct patterns of mRNA expression (FIG. 3b, right panel). The CpG methylation interrogated by the MSP-primers used here largely correlated with reduced gene expression in the case of SFRP1, DKK3, GPX3, COX2/PTGS2, GSTM1, APOD and p57, whereas the detected CpG methylation of DDB2 and HPGD was not accompanied by decreased gene expression. In the case of HPGD the expression was even induced in 3 PCa samples, which clearly showed CpG methylation. These results indicate that CpG methylation of a promoter should not be interpreted as proof for its transcriptional repression.

### Table 3

Summary of the analysis of selected candidate genes up-regulated in PCa.

Experimental results for 50 selected, CpG-island containing genes induced upon treatment with 5Aza-2′dC and TSA in prostate carcinoma cell lines are summarized. Bold gene-symbols represent genes previously known to be silenced by CpG-methylation in human cancer, but not in PCa. Underlined chromosomal locations represent regions of frequent LOH in PCa. Cell line abbreviations in bold indicate usage of gDNA from this cell line for bisulfite sequencing. (L=LNCap; P=PC3; D=Du-145; BS-seq.=bisulfite sequencing analysis; MSP=methylation-specific PCR)

TABLE 3

| | | | | | Methylation detected | | |
|---|---|---|---|---|---|---|---|
| | | | | | by | | |
| | | | | | BS- | | |
| | | | | | seq. | MSP | |
| | | | | | in | | in |
| | | Chr. | Induced | | cell | in cell | primary |
| Gene | Symbol | location | by 5Aza2dC | Function | lines | lines | tumors |
| 14-3-3sigma | SFN | 1p35 | L | G₂/M transition | | yes | 41/41 |
| (stratifin) | | | | | | | |
| secreted frizzled- | SFRP1 | 8p12 | P | Wnt signaling | | yes | 34/41 |
| related protein 1 | | | | | | | |
| apolipoprotein D | APOD | 3q26 | L, P | HDL component | | yes | n.d. |
| tissue factor | TFPI2 | 7q22 | P, D | ECM proteases | | No | |
| pathway inhibitor 2 | | | | inhibitor | | | |
| thrombospondin 1 | THBS1 | 15q15 | L, P, D | angiogenesis | | No | |
| | | | | inhibitor | | | |
| retinoblastoma | RIZ1 | 1p36 | P | methyltransferase | | No | |
| protein- | | | | | | | |
| interacting zinc | | | | | | | |
| finger | | | | | | | |
| caspase 7 | CASP7 | 10q25 | P, D | apoptosis | no | | |
| apoptotic | APAF1 | 12q23 | P, D | apoptosis | no | | |
| protease | | | | | | | |
| activating factor | | | | | | | |
| apoptosis- | APPD | 19q11 | D | apoptosis | no | | |
| inducing protein D | | | | | | | |
| tumor necrosis | TNFRSF10B | 8p22 | D | apoptosis | no | | |
| factor receptor | | | | | | | |
| 10b | | | | | | | |
| cyclin-dependent | CDKN1C | 11p15 | L, P | cdk inhibitor | yes | yes | 23/41 |
| kinase inhibitor | | | | | | | |
| 1C (p57, Kip2) | | | | | | | |
| cyclin-dependent | CDKN2D | 19p13 | P | cdk inhibitor | no | | |
| kinase inhibitor | | | | | | | |
| 2D (p19) | | | | | | | |
| cyclin-dependent | CDKN1A | 6p21.2 | L, P, D | cdk inhibitor | no | | |
| kinase inhibitor | | | | | | | |
| 1A (p21, Cip1) | | | | | | | |
| retinoblastoma- | RBL2 | 16q12 | D | cell cycle | no | | |
| like 2 (p130) | | | | | | | |
| glutathione S- | GSTM1 | 1p13 | L | detoxification | yes | yes | 24^{§}/41 |
| transferase M1 | | | | | | | |
| glutathione | GPX3 | 5q23 | L, P | detoxification | yes | yes | 38/41 |
| peroxidase 3 | | | | | | | |
| HUS1 | HUS1 | 7p13 | D | DNA damage | no | | |
| | | | | response | | | |
| meiotic | MRE11A | 11q21 | D | DNA damage | no | | |
| recombination 11 | | | | response | | | |
| xeroderma | XPC | 3p25 | L | DNA repair | no | | |
| pigmentosum, | | | | | | | |
| complementation | | | | | | | |
| group C | | | | | | | |
| damage-specific | DDB2 | 11p12 | L | DNA repair | yes | yes | 34/41 |
| DNA binding | | | | | | | |
| protein 2 | | | | | | | |
| postmeiotic | PMS2 | 7p22 | D | DNA repair | no | | |
| segregation | | | | | | | |
| increased 2 | | | | | | | |
| breast cancer 2, | BRCA2 | 13q12.3 | D | DNA repair, | no | | |
| early onset | | | | transcription | | | |
| cylindromatosis | CYLD | 16q11 | P | deubiquitination | no | | |
| (turban tumor | | | | | | | |
| syndrome) | | | | | | | |
| bridging | BIN1 | 2q14 | D | differentiation | low | n.d. | n.d. |
| integrator 1 | | | | | | | |
| growth arrest | GADD45A | 1p31.2 | P, D | growth arrest | low | n.d. | n.d. |
| and DNA- | | | | | | | |
| damage- | | | | | | | |
| inducible, alpha | | | | | | | |
| connective tissue | CTGF | 6q23 | P, D | growth factor | no | | |
| growth factor | | | | | | | |
| nerve growth | NGFR | 17q21 | P, D | growth factor | no | | |
| factor receptor | | | | receptor | | | |
| interferon | IRF1 | 5q31 | P, D | interferon response | no | | |
| regulatory factor 1 | | | | | | | |
| interferon | IRF7 | 11p15 | L, P, D | interferon response | no | | |
| regulatory factor 7 | | | | | | | |
| hydroxyprostaglandin | HPGD | 4q34 | L, P | prostaglandin | yes | yes | 30/41 |
| dehydrogenase | | | | signaling | | | |
| 15-(NAD) | | | | | | | |
| prostaglandin E | PTGER4 | 5p13 | L, P, D | prostaglandin | no | | |
| receptor 4 | | | | signaling | | | |
| (subtype EP4) | | | | | | | |
| cyclooxygenase 2 | COX2/PTGS2 | 1q25 | L, P | prostaglandin | yes | yes | 32/41 |
| | | | | signaling | | | |
| sequestosome 1 | SQSTM1 | 5q35 | L, P | protein degradation | no | | |
| dual specificity | DUSP1 | 5q34 | P, D | signaling | no | | |
| phosphatase 1 | | | | | | | |
| jun B protooncogene | JUNB | 14q32 | P, D | transcription factor | no | | |
| cut-like 2 | CUTL2 | 12q24 | P | transcription factor | yes | n.d. | n.d. |
| zinc finger | ZFP36 | 19q13 | L, D | transcription factor | no | | |
| protein 36, C3H | | | | | | | |
| type, homolog | | | | | | | |
| Cbp/p300- | CITED2 | 6q23 | P, D | transcriptional | no | | |
| interacting | | | | control | | | |
| transactivator | | | | | | | |
| SWI/SNF related | SMARCA1 | Xq25 | D | transcriptional | no | | |
| regulator of | | | | control | | | |
| chromatin A1 | | | | | | | |
| inhibitor of DNA | ID3 | 1p36 | L, P | transcriptional | no | | |
| binding 3 | | | | control | | | |
| serum-inducible | SNK | 5q12 | L, P | signaling | no | | |
| kinase | | | | | | | |
| serum/glucocorticoid | SGK | 6q23 | L, P, D | signaling | no | | |
| regulated | | | | | | | |
| kinase | | | | | | | |
| serine/threonine | STK38L | 12p12.3 | L | signaling | no | | |
| kinase 38 like | | | | | | | |
| growth arrest- | GAS2L1 | 22q12.2 | L | unknown | no | | |
| specific 2 like 1 | | | | | | | |
| deleted in liver | DLC1 | 8p22 | D | unknown | no | | |
| cancer 1 | | | | | | | |
| ras-induced | RIS1 | 3p.21 | D | unknown | yes | yes | n.d. |
| senescence 1 | | | | | | | |
| B-cell | BTG1 | 12q22 | D | unknown | no | | |
| translocation | | | | | | | |
| gene 1, anti- | | | | | | | |
| proliferative | | | | | | | |
| B-cell | BTG3 | 21q21 | P, D | unknown | no | | |
| translocation | | | | | | | |
| gene 3, anti- | | | | | | | |
| proliferative | | | | | | | |
| dickkopf homolog 3 | DKK3 | 11p15 | P | Wnt signaling | yes | yes | 28/41 |
| dickkopf homolog 1 | DKK1 | 10q11 | D | Wnt signaling | no | | |

### In Vivo Methylation of Tumor Suppressive Genes in PCa

The in vivo CpG-methylation status of the genes identified having CpG-methylation in PCa cell lines was determined in 41 primary PCa samples obtained after radical (37 cases) or transurethral (4 cases) resection. In addition, the genes DDB2 and HPGD were included in this analysis, although we had not detected a correlation between CpG methylation and down-regulation of mRNA expression for these genes. Nonetheless, the detection of PCa-specific CpG methylation in the promoter of these genes may be useful for diagnostic applications. Resected prostate tissue containing PCa also includes a number of other cell types, such as stromal cells, infiltrating T-cells and prostate epithelial cells of normal glands. Therefore, laser-microdissection was employed to isolate primary PCa from paraffin-embedded sections obtained from 41 patients. Furthermore, non-neoplastic prostate epithelial cells were isolated from samples obtained from 9 patients with benign prostate hyperplasia, which did not present PCa and were in the age-group as the 41 PCa patients. Isolated genomic DNA from these samples was subjected to MSP analysis (FIGS. 4a,b and d). The analyzed genes showed CpG-methylation at medium to high frequencies (56-100%; Table 3, right column). The highest frequency of CpG-methylation was detected for the 14-3-3σ gene, which showed CpG-methylation in all 41 primary tumor samples analyzed. The 14-3-3σ promoter was not CpG-methylated in non-neoplastic prostate epithelial cells isolated by laser-microdissection from sections derived from 4 different patients (FIG. 4a,b). However, one patient did show a low level of CpG-methylation of the 14-3-3σ promoter in epithelial cells of hyperplastic prostatic glands (FIG. 4a, b). An explanation for this observation could be that this patient had an undetected PCa precursor lesion. In support of this notion, this DNA sample also revealed CpG-methylation of the GPX3 gene, which was not CpG-methylated in the 4 other non-neoplastic samples (FIG. 4a). CpG-methylation of 14-3-3σ therefore seems to be a highly PCa-specific event.

CpG-methylation was detected for SFRP1 in 34 (83%), for COX2/PTGS2 in 32 (78%) and for DKK3 in 28 (68%), GPX3 in 38 (93%), p57 in 23 (56%), HPGD in 30 (73%) and DDB2 in 34 (83%) cases of the 41 PCa samples analyzed. Predominant methylation of GSTM1 was detected in 24 (58%) of 41 cases. The silencing of 14-3-3σ in the set of 41 PCa analyzed here is depicted for comparison (FIG. 4d). In non-neoplastic prostate epithelial cells only un-methylated alleles were detected for these genes, suggesting that the CpG-methylation of these genes is highly specific for neoplastic prostate epithelial cells and could therefore represent diagnostic advantages over the other genes identified here.

For SFRP and COX2/PTGS2 no CpG methylation was detected in BPH derived from 9 different patients, suggesting that the CpG methylation of these genes is specific for neoplastic prostate epithelial cells. CpG methylation was detected for GPX3 in two and for DKK3 in one of 9 analyzed BPH samples (FIG. 4d).

For GSTM1 CpG methylation was detected in the 5 BPH samples analyzed (FIG. 4d). However, the CpG methylation of GSTM1 was elevated in the majority of PCa samples, whereas in the non-neoplastic BPH cells equal signals for the PCR-products representing methylated and unmethylated GSTM1 alleles were detected.

The CpG-methylation of GSTM1 did not show a clear qualitative difference between PCa and non-neoplastic cells, since most non-neoplastic samples also gave rise to a CpG-methylation-specific signal (FIG. 4a). However, the ratio was clearly shifted towards the CpG-methylated allele of GSTM1 in the PCa samples, whereas the non-neoplastic cells showed equal signals for both PCR-products. Furthermore, the un-methylated allele was not detected in several samples of PCa. For diagnostic purposes and to elaborate our findings, a quantitative analysis of GSTM1 CpG-methylation may be used. The pattern of CpG-methylation of HPGD and DDB2 indicates that their CpG-methylation may be related to other biological processes, such as differentiation, but not necessarily provide a selective advantage during PCa development. For example 2 of the non-neo-plastic samples showed complete CpG-methylation of HPGD.

In order to evaluate the tumor-specificity of the CpG methylation detected in PCa we examined the CpG methylation status in human diploid fibroblasts (HDF) derived from neonatal skin, in stroma isolated from 5 cancer-free prostate specimens and in peripheral blood mononuclear cells (PBMC) from 6 individuals (FIG. 4d). The p57 gene did not show CpG methylation in stromal cells. Partial CpG methylation of SFRP1 and COX2/PTGS2 was evident only in one of five analyzed stroma samples. One of six PBMC samples showed partial methylation of SFRP1 and p57 genes. By contrast, DKK3 and GPX3 displayed CpG methylation in most of the stromal samples and blood cells.

The high frequency of CpG-methylation observed in primary tumors shows that the differences in CpG-methylation initially detected in PCa cell lines did not result from prolonged in vitro passaging of PCa cell lines, but instead reflect tumor-specific events. Furthermore, the frequencies of CpG-methylation detected in a representative number of cases (41 primary PCa and 6 PCa cell lines) and their absence in non-neoplastic prostate epithelial cells imply that these alterations in CpG-methylation play an important role during PCa development and may be relevant for diagnostic and/or therapeutic purposes.

### Loss of SFRP1 Expression in Pca

In order to determine whether the CpG methylation of SFRP1 affects the expression of the respective gene product in primary tumors, the level of SFRP1 protein expression was determined by immunohistochemistry in PCa samples derived from 39 different patients (representative example shown in FIG. 5a). In non-neoplastic prostate glands most of the luminal cells were positive for SFRP1 with a characteristic granular cytoplasmic and apical membrane staining, whereas PCa cells were devoid of SFRP1 staining. A prominent down-regulation (>50% of reduction) or complete loss of SFRP1 protein was detected in 29 of 39 PCa samples (data not shown).

### Functional Analysis of DKK3 and SFRP1

Epigenetic inactivation of the members of the SFRP gene family presumably contributes to activation of WNT signaling in colorectal cancer (16). DKK3 negatively regulates the β-catenin pathway in osteosarcoma cells (21). Therefore, we asked whether silencing of SFRP1 and/or DKK3 expression in prostate carcinoma cells is associated with constitutive activation of WNT/β-catenin signaling. Unexpectedly, β-catenin was localized at the cell membrane and absent from the nucleus in PC3 and Du145 cells (FIG. 5b), which show significant silencing of DKK3 and SFRP1 (FIG. 3a). As nuclear β-catenin is a hallmark of an activated WNT/1-catenin pathway, this pathway is presumably not active in PCa. Furthermore, co-transfection with a WNT1 expression construct did not result in activation of a TCF-reporter in PC3 and Du145 cells (FIG. 5c). In contrast, HCT116 colon cancer cell lines, which harbor an activated WNT/APC/TCF4 pathway (22), showed nuclear β-catenin localization (data not shown) and strong activation of the wildtype but not the mutant TCF reporter by WNT1 co-expression (FIG. 5c). Recently, it has been reported that WNT1 activates the MAP kinase pathway (23). Therefore, we tested whether the WNT antagonists DKK3 and SFRP1 inhibit the MAP kinase pathway. In exponentially growing PC3 cells ectopically expressing DKK3 the level of ERK1 and ERK2 phosphorylation, which is indicative of MAP kinase activity, was diminished (FIG. 11a). Furthermore, PC3 cells ectopically expressing DKK3 or SFRP1 showed a significant decrease in colony formation (FIG. 11b), which was due to a decrease in colony size. The rate of spontaneous apoptosis was not affected by ectopic expression of DKK3 or SFRP1 as determined by flow cytometry (data not shown). Therefore, loss of DKK3 and SFRP1 expression by epigenetic silencing presumably promotes the proliferation of prostate epithelial cells.

### Loss of 14-3-3σ Expression in PCa

By Western blot analysis an inverse correlation between the degree of CpG-methylation and protein expression was identified (FIG. 8): LNCaP cells, which display complete CpG-methylation of 14-3-3σ were devoid of 14-3-3σ expression. PPC-1 cells, which have CpG-methylated and un-methylated 14-3-3σ alleles, show a significant down-regulation of 14-3-3σ protein expression. The cell lines Du-145, PC3 and TSU-Pr1 did not reveal any CpG-methylation in the 14-3-3σ gene and showed relatively high levels of 14-3-3σ protein expression. The highest level of 14-3-3σ expression was detected in the PrECs, which lack CpG-methylation of the 14-3-3σ gene. Interestingly, the cell lines Du-145 and PC3 harbor p53 mutations, whereas LNCaP cells express wild-type p53. This correlation suggests that silencing of 14-3-3σ may potentially alleviate the requirement to inactivate p53 in Pca.

The in vivo CpG-methylation status of 14-3-3σ was determined in primary PCa samples obtained after radical prostatectomy. Resected prostate tissue containing PCa also includes a number of other cell types, such as stromal cells, infiltrating T-cells and prostate epithelial cells of normal glands. Since these cells are in close proximity to or overlap with areas of PCa, they would obscure the analysis of PCa-specific CpG-methylation in case DNA is extracted from larger areas surrounding the cancer tissue. Therefore, laser-microdissection was employed to specifically isolate primary PCa cells from paraffin-embedded sections obtained from 41 patients. Furthermore, corresponding non-neoplastic prostate epithelial cells were isolated from 10 of these samples. Genomic DNA obtained from the isolated cells was subjected to MSP analysis. In the PCa cells 14-3-3σ showed CpG-methylation at medium to high degrees (FIG. 9a): in three cases only the methylated allele was detected and, with the exception of one case, the PCR-product representing the methylated allele was more prominent than the PCR-product specific for the un-methylated allele. The adjacent, normal prostate epithelial cells did not show CpG-methylation of 14-3-3σ (FIG. 9b). This finding strongly supports the PCa-specific nature of this epigenetic alteration. In stromal cells of the prostate we found a prominent CpG-methylation of 14-3-3σ, which underscores the necessity of lasermicro-dissection for these analyses (FIG. 9c). Furthermore, hyperproliferative prostate epithelial cells obtained from 5 patients with benign prostate hyperplasia (BPH), which did not present PCa and were in the same age-group as the 41 PCa patients, were subjected to MSP-analysis. The 14-3-3σ promoter was not CpG-methylated in prostate epithelial cells in 4 cases of BPH (FIG. 9d). In a fifth BPH specimen a weak signal representing a CpG-methylated 14-3-3σ allele was detected (FIG. 9d). The high frequency of CpG-methylation at the 14-3-3σ locus observed in primary tumors shows that the CpG-methylation initially detected in PCa cell lines did not result from prolonged in vitro passaging of PCa cell lines, but reflects a carcinoma-specific event. This notion is further supported by the finding that benign, but hyperproliferative BPH shows no significant 14-3-3σ silencing.

In order to determine whether CpG-methylation of the 14-3-3σ gene affects the expression of the corresponding gene product in vivo, the level of 14-3-3σ expression was determined by immuno-histochemistry in tissue sections of the prostate. 41 different PCa samples were analyzed with an affinity purified antibody specific for the 14-3-3σ protein. In normal basal and luminal prostate epithelial cells and in prostate epithelial cells representing BPH and atrophic lesions an intense, cytoplasmic staining for 14-3-3σ protein was detected (FIG. 10a-f). The expression of 14-3-3σ protein was down-regulated markedly (>50%) in neoplastic cells and glands of 26 PCa samples. Representative examples are shown in FIGS. 3a and 3b. 12 specimen showed a moderate reduction (examples in FIG. 10d,e) and 3 a minor decrease in 14-3-3σ protein specific staining (data not shown). We also detected down-regulation of 14-3-3σ protein expression in PIN (prostatic intraepithelial neoplasia) lesions, which represent precursors of PCa (FIG. 10e, f).

### 3. Discussion

Gene inactivation by epigenetic silencing plays a pivotal role in tumor development⁹. However, the identification of tumor suppressive genes inactivated via this mechanism is far from complete. Therefore, we set out to analyse the genome-wide methylation-status of PCa by a recently described approach, which combines pharmacological reversion of epigenetic silencing and micro-array analysis^{21,22}. Based on the function of the re-expressed gene product and the presence of CpG-islands, we chose 50 genes for further analysis. Since CpG-methylation was confirmed for 12 of 50 re-expressed genes, which contained CpG-islands, only a fraction (<24%) of the genes re-expressed after treatment with 5Aza-2′dC and TSA contain CpG-methylated promoter regions. 9 of the genes also showed a high frequency of CpG-methylation in primary PCa samples. For 2 of these genes, down-regulation or loss of protein expression was analysed and confirmed in primary PCa.

DNA damage binding protein 2 (DDB2) is an important component of the nucleotide excision repair system. It can be induced by p53 and is involved in the repair of UV-induced DNA damage³¹. Furthermore, DDB2 is mutated in xeroderma pigmentosum E group cells³². Knock-out mice display enhanced skin cancerogenesis induced after UV exposure³³. We have observed re-expression of DDB2 mRNA in the LNCaP cell line after de-methylating treatment and dense CpG-methylation in the promoter of this gene. However, MSP analysis revealed that DDB2 is also CpG-methylated in PrECs and in all normal prostate samples analyzed. Nevertheless, quantitative differences in CpG-methylation may also lead to tumor-specific down-regulation. As determined by MSP analysis, the CpG-methylation of DDB2 seems to be more pronounced in the PCa cell lines than in PrECs.

Glutathione peroxidase 3 (GPX3) catalyzes the reduction of peroxides by glutathione and functions in the protection of cells against oxidative damage. Its down-regulation may lead to an impaired defense against endogenous and exogenous genotoxic compounds, similar to the role proposed for GSTP1 and GSTM1. We identified GPX3 as a target for epigenetic silencing in PCa both in vitro and in vivo. The high frequency of GPX3 silencing (93%) detected in primary PCa suggests that alterations in this detoxification pathway are critical for prostate tumorigenesis.

The tumor-specific hypermethylation of GSTM1 identified here, explains the decreased expression of GSTM1 in PCa versus normal prostate tissue reported in two previous studies^{34,35}. Down-regulation of GSTM1 was also shown in colorectal carcinoma³⁶. Consistent with a general role of GSTM1 silencing in tumorigenesis, we detected CpG-methylation of GSTM1 in several breast cancer cell lines. Glutathione S-transferase μ 1 (GSTM1) belongs to a family of enzymes that catalyze the conjugation of reduced glutathione to a variety of electrophiles. GSTM1 detoxifies mutagens, mainly epoxides formed from common carcinogens such as polycyclic aromatic hydrocarbons, and thus may play a protective role for the genome, as was proposed for GSTP1. The GSTM1 gene shows polymorphisms in several allelic variants and GSTM1 loss has been implicated in lung cancer³⁷. However, no significant association of GSTM1 polymorphisms or deletion with PCa have been reported. The tumor-specific hypermethylation of GSTM1 identified here may explain the decreased expression of GSTM1 in PCa detected in three previous studies (34, 35).

Two inhibitors of wnt-signalling were identified in this study: SFRP1 and DKK3. The protein product of the Secreted Frizzled-related protein 1 (SFRP1) gene contains a cysteine-rich domain similar to the wnt-binding site of Frizzled receptors and negatively regulates the wnt pathway, which is frequently activated by mutations in the APC and β-catenin genes³⁸. Here we show that expression of SFRP1 is markedly reduced or lost in ˜80% of primary PCa by CpG-methylation of the SFRP1 promoter. Frequent hypermethylation of SFRP1 as well as of other members of the SFRP gene family in colorectal carcinomas was detected in colorectal cancer²¹. Interestingly, the SFRP1 gene is located on chromosome 8p12, a region which frequently undergoes LOH in PCa. Recently, SFRP1 was shown to undergo both genetic and epigenetic alterations in colon and bladder cancer^{39,40}. Interestingly, we have previously observed an induction of SFRP1 mRNA in human primary fibroblasts undergoing replicative senescence, a process which is considered to be a tumor-suppressive mechanism⁴¹. Taken together these results argue for an important role of SFRP1 down-regulation in prostate tumorigenesis.

Dickkopf 3 (DKK3) is a morphogen which regulates the wnt-pathway. The zonal distribution of DKK3 expression in the adrenal gland suggests that it could be involved in zonal differentiation or growth⁴². Our data suggest that the WNT/β-catenin pathway is not activated in PCa cell lines (Du-145 and PC3) with silenced SFRP1 and DKK3 genes. This is in agreement with a recent comprehensive study of 101 cases of primary PCa: none of the tumors showed nuclear β-catenin staining (63). However, genetic alterations of β-catenin or APC were detected in a subset of advanced PCa and were associated with an resistance to apoptosis (64) (65). Loss of SFRP1 and DKK3 expression may activate alternative signaling pathways. Recently, it was reported that transactivation of the EGF receptor by WNT ligands, which results in MAP-kinase activation, is inhibited by SFRP1 and DKK1 (66). Our data indicate that DKK3 may also have an inhibitory effect on MAP kinase signaling. The epigenetic silencing of DKK3 by CpG-methylation observed here may therefore contribute to the dedifferentiation of PCa cells.

p57/KIP2 belongs to a family of conserved CDK inhibitors, which negatively regulate the cell cycle. Ectopic expression of p57 suppresses cell transformation by inhibiting CDKs and interaction with proliferating cell nuclear antigen⁴³, whereas cells lacking p57 show increased cell proliferation and decreased differentiation^{44,45}. The p57 gene is located on chromosome 11p15.5, a region implicated in both sporadic cancers and the Beckwith-Wiedemann syndrome, a familial cancer syndrome. Mutated forms of p57 have rarely been detected in human tumors⁴⁶. CpG-methylation of p57 associated with diminished expression was shown in several tumor types: gastric, hepatocellular, pancreatic carcinomas and acute myeloid leukaemia^{47 48}. The p57 gene is located in the vicinity of imprinted genes (IGF2 and H19) and itself displays features of an imprinted gene. The maternal allele is preferentially expressed; however, the imprinting is not absolute, as the paternal allele is also expressed at low levels in most tissues⁴⁹. Furthermore, the relevance of DNA methylation for the imprinting of p57 is not clear, as CpG-methylation has not been detected in the 5′ region of p57 in normal tissue^{48,50}. Consistently, we could not detect CpG-methylation of p57 in normal PrECs. These results suggest, that the tumor-specific CpG-methylation of p57 is not due to imprinting but a specific feature of Pca.

COX2/PTGS2, or cyclooxygenase 2 (COX-2), is an inducible enzyme catalyzing the synthesis of prostaglandin H2, which is a precursor of other prostanoids playing an important role in inflammation and, possibly, in carcinogenesis reviewed in Ref.⁵¹. Overexpression of COX2/PTGS2 was found in several tumor types including PCa⁵². Nonsteroidal anti-inflammatory drugs (NSAIDs) decrease the risk of developing PCa⁵³ and inhibit the growth of PCa in a xenograft model⁵⁴. However, the effects of NSAIDs on cancer cells may not be caused by inhibition of COX-2. Zha et al. found no consistent overexpression of COX-2 in PCa or high-grade prostatic intraepithelial neoplasia compared to adjacent normal prostate tissue⁵⁵. Instead, high COX-2 levels were detected in areas of proliferative inflammatory atrophy, which presumably represents an early precursor of PCa³. Interestingly, expression of COX-2 protein was not detected in PCa cell lines LNCaP, PC3 and Du-145, which is consistent with the silencing of the COX-2 gene by CpG-methylation reported here. According to our analysis the CpG methylation of SFRP1, COX2/PTGS2 and p57 has the highest specificity for PCa and is therefore of higher relevance for potential diagnostic applications. However, these results require validation in larger cohorts of patients in the future.

Consistent with our data, CpG-methylation of the promoter region of COX2/PTGS2 and absence of COX-2 expression was also detected in a subset of gastric⁴⁸ and colorectal cancers⁵⁶. Irrespective of its influence on gene expression, the high frequency of tumor specific CpG-methylation of the COX2/PTGS2 gene detected in PCa in this study implicates CpG-methylation of COX2/PTGS2 as a potential diagnostic marker for Pca.

HPGD is the key enzyme of prostaglandin degradation. By catalyzing the conversion of the 15-hydroxyl group of prostaglandins into a keto group, this ubiquitous enzyme strongly reduces the biologic activity of prostaglandins. Prostanoids and their receptors, especially EP receptors, have been implicated in tumor development and growth⁵¹. However, it is not known whether the altered rate of ligand degradation may contribute to these effects. Induction of HPGD activity was associated with inhibition of tumor growth⁵⁷. Down-regulation of HPGD expression was observed in esophageal squamous cell carcinoma cell line upon gain of metastatic potential⁵⁸. We detected CpG-methylation of HPGD in PCa cell lines but not in PrECs. However, 4 of 5 specimens of normal prostate showed CpG-methylation of HPGD, although tumor samples showed variable degrees of methylation.

As discussed by Laird, detection of aberrant CpG-methylation has several significant advantages when compared to protein- or RNA-based tumor markers⁵⁹. Since 14-3-3σ, SFRP1, COX2/PTGS2 and GSTM1 are hypermethylated in a number of other carcinomas a combination with the PCa-specific CpG-methylation markers (GPX3, DKK3) identified in this study may allow the development of a highly sensitive and specific diagnostic assay. As changes in DNA-methylation seem to occur early in carcinogenesis, this assay may be especially suited to detect cells or free DNA derived from early PCa lesions in body fluids.

From other types of cancer it is known that CpG-methylation of 14-3-3σ may represent an early event during tumor progression. For example in breast cancer it has been shown that CpG-methylation of 14-3-3σ occurs during the transition from atypical hyperplastic lesions to carcinoma in situ⁶². The loss of 14-3-3σ protein expression in PIN lesions indicates that 14-3-3σ inactivation is an early event during PCa progression.

Of all 14-3-3σ proteins, the 14-3-3σ isoform is most evidently implicated in human cancer³⁰. After DNA damage, 14-3-3σ is transcriptionally induced by p53. Experimental inactivation of 14-3-3σ in colorectal carcinoma cells leads to impairment of the G₂/M cell cycle checkpoint and increased genomic instability. CpG-hypermethylation of 14-3-3σ and loss of 14-3-3σ expression has been detected in a number of different types of carcinomas³⁰. Recently, we found 14-3-3σ inactivation by CpG-methylation in 28 of 41 (68%) cases of basal cell carcinoma of the skin²⁹. The general CpG-methylation of 14-3-3σ described here for PCa represents the highest frequency of 14-3-3σ silencing which has been observed in any type of carcinoma so far and may indicate an absolute requirement of 14-3-3σ down-regulation for PCa formation. The strategy used to identify 14-3-3σ as a gene specifically silenced in PCa, i.e. reversion of CpG-methylation mediated gene-repression in PCa cell lines, clearly argues for a role of CpG-methylation in the down-regulation/loss of 14-3-3σ at the level of mRNA and protein expression. Previously, it was shown that ectopic expression of 14-3-3σ by adenoviral infection in primary prostate epithelial cells induces a G2/M arrest⁶⁰. Deletion of 14-3-3σ in epithelial cells promotes genetic instability³⁰. The silencing of 14-3-3σ expression observed here may therefore contribute to PCa formation by accelerating the inactivation of additional tumor suppressive genes or activation of oncogenes. Alternatively, 14-3-3σ may have additional tumor suppressive functions.

Detection of aberrant CpG-methylation has several significant advantages when compared to protein- or RNA-based tumor markers⁶². Since 14-3-3σ is also hypermethylated in a number of other carcinomas a combination with PCa-specific CpG-methylation markers may allow the development of a highly sensitive and specific diagnostic assay in the future. As changes in DNA-methylation seem to occur early in carcinogenesis, this assay may be especially suited to detect cells or free DNA derived from early PCa lesions in body fluids.

### REFERENCES

1. Jemal, A. et al. Cancer statistics, 2004. CA Cancer J Clin 54, 8-29 (2004).
2. Sakr, W. A. et al. High grade prostatic intraepithelial neoplasia (HGPIN) and prostatic adenocarcinoma between the ages of 20-69: an autopsy study of 249 cases. In Vivo 8, 439-43 (1994).
3. Nelson, W. G., De Marzo, A. M. & Isaacs, W. B. Prostate cancer. N Engl J Med 349, 366-81 (2003).
4. Abate-Shen, C. & Shen, M. M. Molecular genetics of prostate cancer. Genes Dev 14, 2410-34 (2000).
5. Karan, D., Lin, M. F., Johansson, S. L. & Batra, S. K. Current status of the molecular genetics of human prostatic adenocarcinomas. Int J Cancer 103, 285-93 (2003).
6. Suzuki, H. et al. Interfocal heterogeneity of PTEN/MMAC1 gene alterations in multiple metastatic prostate cancer tissues. Cancer Res 58, 204-9 (1998).
7. Schulz, W. A., Burchardt, M. & Cronauer, M. V. Molecular biology of prostate cancer. Mol Hum Reprod 9, 437-48 (2003).
Kote-Jarai, Z. et al. Relationship between glutathione S-transferase M1, P1 and T1 polymorphisms and early onset prostate cancer. Pharmacogenetics 11, 325-30 (2001).
9. Feinberg, A. P. & Tycko, B. The history of cancer epigenetics. Nat Rev Cancer 4, 143-53 (2004).
10. Jones, P. A. & Baylin, S. B. The fundamental role of epigenetic events in cancer. Nat Rev Genet 3, 415-28 (2002).
11. Hopkins-Donaldson, S. et al. Silencing of death receptor and caspase-8 expression in small cell lung carcinoma cell lines and tumors by DNA methylation. Cell Death Differ 10, 356-64 (2003).
12. Soengas, M. S. et al. Inactivation of the apoptosis effector Apaf-1 in malignant melanoma. Nature 409, 207-11 (2001).
13. Rhee, I. et al. DNMT1 and DNMT3b cooperate to silence genes in human cancer cells. Nature 416, 552-6 (2002).
14. Sansom, O. J. et al. Deficiency of Mbd2 suppresses intestinal tumorigenesis. Nat Genet 34, 145-7 (2003).
15. Singal, R., van Wert, J. & Bashambu, M. Cytosine methylation represses glutathione S-transferase P1 (GSTP1) gene expression in human prostate cancer cells. Cancer Res 61, 4820-6 (2001).
16. Kuzmin, I. et al. The RASSF1A tumor suppressor gene is inactivated in prostate tumors and suppresses growth of prostate carcinoma cells. Cancer Res 62, 3498-502 (2002).
17. Nakayama, T. et al. The role of epigenetic modifications in retinoic acid receptor beta2 gene expression in human prostate cancers. Lab Invest 81, 1049-57 (2001).
18. Harden, S. V. et al. Quantitative GSTP1 methylation and the detection of prostate adenocarcinoma in sextant biopsies. J Natl Cancer Inst 95, 1634-7 (2003).
19. Cameron, E. E., Bachman, K. E., Myohanen, S., Herman, J. G. & Baylin, S. B. Synergy of demethylation and histone deacetylase inhibition in the re-expression of genes silenced in cancer. Nat Genet 21, 103-7 (1999).
20. Paz, M. F. et al. A systematic profile of DNA methylation in human cancer cell lines. Cancer Res 63, 1114-21 (2003).
21. Suzuki, H. et al. A genomic screen for genes upregulated by demethylation and histone deacetylase inhibition in human colorectal cancer. Nat Genet 31, 141-9 (2002).
22. Yamashita, K. et al. Pharmacologic unmasking of epigenetically silenced tumor suppressor genes in esophageal squamous cell carcinoma. Cancer Cell 2, 485-95 (2002).
23. Herman, J. G., Graff, J. R., Myohanen, S., Nelkin, B. D. & Baylin, S. B. Methylation-specific PCR: a novel PCR assay for methylation status of CpG islands. Proc Natl Acad Sci USA 93, 9821-6 (1996).
24. Karpf, A. R. & Jones, D. A. Reactivating the expression of methylation silenced genes in human cancer. Oncogene 21, 5496-503 (2002).
25. Du, Y. et al. Hypermethylation in human cancers of the RIZ1 tumor suppressor gene, a member of a histone/protein methyltransferase superfamily. Cancer Res 61, 8094-9 (2001).
26. Li, Q., Ahuja, N., Burger, P. C. & Issa, J. P. Methylation and silencing of the Thrombospondin-1 promoter in human cancer. Oncogene 18, 3284-9 (1999).
27. Konduri, S. D. et al. Promoter methylation and silencing of the tissue factor pathway inhibitor-2 (TFPI-2), a gene encoding an inhibitor of matrix metalloproteinases in human glioma cells. Oncogene 22, 4509-16 (2003).
28. Karpf, A. R., Moore, B. C., Ririe, T. O. & Jones, D. A. Activation of the p53 DNA damage response pathway after inhibition of DNA methyltransferase by 5-aza-2′-deoxycytidine. Mol Pharmacol 59, 751-7 (2001).
29. Lodygin, D., Yazdi, A. S., Sander, C. A., Herzinger, T. & Hermeking, H. Analysis of 14-3-3sigma expression in hyperproliferative skin diseases reveals selective loss associated with CpG-methylation in basal cell carcinoma. Oncogene 22, 5519-24 (2003).
30. Hermeking, H. The 14-3-3 cancer connection. Nat Rev Cancer 3, 931-43 (2003).
31. Tang, J. Y., Hwang, B. J., Ford, J. M., Hanawalt, P. C. & Chu, G. Xeroderma pigmentosum p48 gene enhances global genomic repair and suppresses UV-induced mutagenesis. Mol Cell 5, 737-44 (2000).
32. Itoh, T., Mori, T., Ohkubo, H. & Yamaizumi, M. A newly identified patient with clinical xeroderma pigmentosum phenotype has a non-sense mutation in the DDB2 gene and incomplete repair in (6-4) photoproducts. J Invest Dermatol 113, 251-7 (1999).
33. Itoh, T., Cado, D., Kamide, R. & Linn, S. DDB2 gene disruption leads to skin tumors and resistance to apoptosis after exposure to ultraviolet light but not a chemical carcinogen. Proc Natl Acad Sci USA 101, 2052-7 (2004).
34. Vanaja, D. K., Cheville, J. C., Iturria, S. J. & Young, C. Y. Transcriptional silencing of zinc finger protein 185 identified by expression profiling is associated with prostate cancer progression. Cancer Res 63, 3877-82 (2003).
35. Chetcuti, A. et al. Identification of differentially expressed genes in organ-confined prostate cancer by gene expression array. Prostate 47, 132-40 (2001).
36. Barker, H. J., Alpert, L. C., Compton, C. C., Maslen, A. & Kirby, G. M. Loss of glutathione S-transferase (GST) mu phenotype in colorectal adenocarcinomas from patients with a GSTM1 positive genotype. Cancer Lett 177, 65-74 (2002).
37. Norppa, H. Genetic susceptibility, biomarker responses, and cancer. Mutat Res 544, 339-48 (2003).
38. Kinzler, K. W. & Vogelstein, B. Lessons from hereditary colorectal cancer. Cell 87, 159-70 (1996).
39. Caldwell, G. M. et al. The Wnt antagonist sFRP1 in colorectal tumorigenesis. Cancer Res 64, 883-8 (2004).
40. Stoehr, R. et al. Deletions of chromosome 8p and loss of sFRP1 expression are progression markers of papillary bladder cancer. Lab Invest (2004).
41. Lodygin, D., Menssen, A. & Hermeking, H. Induction of the Cdk inhibitor p21 by LY83583 inhibits tumor cell proliferation in a p53-independent manner. J Clin Invest 110, 1717-27 (2002).
42. Suwa, T., Chen, M., Hawks, C. L. & Hornsby, P. J. Zonal expression of dickkopf-3 and components of the Wnt signalling pathways in the human adrenal cortex. J Endocrinol 178, 149-58 (2003).
43. Watanabe, H. et al. Suppression of cell transformation by the cyclin-dependent kinase inhibitor p57KIP2 requires binding to proliferating cell nuclear antigen. Proc Natl Acad Sci USA 95, 1392-7 (1998).
44. Yan, Y., Frisen, J., Lee, M. H., Massague, J. & Barbacid, M. Ablation of the CDK inhibitor p57Kip2 results in increased apoptosis and delayed differentiation during mouse development. Genes Dev 11, 973-83 (1997).
45. Zhang, P. et al. Altered cell differentiation and proliferation in mice lacking p57KIP2 indicates a role in Beckwith-Wiedemann syndrome. Nature 387, 151-8 (1997).
46. Tokino, T. et al. Characterization of the human p57KIP2 gene: alternative splicing, insertion/deletion polymorphisms in VNTR sequences in the coding region, and mutational analysis. Hum Genet 97, 625-31 (1996).
47. Shin, J. Y., Kim, H. S., Park, J., Park, J. B. & Lee, J. Y. Mechanism for inactivation of the KIP family cyclin-dependent kinase inhibitor genes in gastric cancer cells. Cancer Res 60, 262-5 (2000).
48. Kikuchi, T. et al. Aberrant methylation and histone deacetylation of cyclooxygenase 2 in gastric cancer. Int J Cancer 97, 272-7 (2002).
49. Matsuoka, S. et al. Imprinting of the gene encoding a human cyclin-dependent kinase inhibitor, p57KIP2, on chromosome 11p15. Proc Natl Acad Sci USA 93, 3026-30 (1996).
50 Chung, W. Y., Yua L., & Tyck Chromosome 11p15.5 regional imprinting: comparative analysis of KIP2 and H19 in human tissues and Wilms' tumors. Hum Mol Genet 5, 1101-8 (1996).
51. Dannenberg, A. J. & Subbaramaiah, K. Targeting cyclooxygenase-2 in human neoplasia: rationale and promise. Cancer Cell 4, 431-6 (2003).
52. Gupta, S., Srivastava, M., Ahmad, N., Bostwick, D. G. & Mukhtar, H. Over-expression of cyclooxygenase-2 in human prostate adenocarcinoma. Prostate 42, 73-8 (2000).
53. Nelson, J. E. & Harris, R. E. Inverse association of prostate cancer and non-steroidal anti-inflammatory drugs (NSAIDs): results of a case-control study. Oncol Rep 7, 169-70 (2000).
54. Liu, X. H. et al. Inhibition of cyclooxygenase-2 suppresses angiogenesis and the growth of prostate cancer in vivo. J Urol 164, 820-5 (2000).
55. Zha, S. et al. Cyclooxygenase-2 is up-regulated in proliferative inflammatory atrophy of the prostate, but not in prostate carcinoma. Cancer Res 61, 8617-23 (2001).
56. Toyota, M. et al. Aberrant methylation of the Cyclooxygenase 2 CpG island in colorectal tumors. Cancer Res 60, 4044-8 (2000).
57. Quidville, V. et al. Tumor growth inhibition by indomethacin in a mouse model of human medullary thyroid cancer: implication of cyclooxygenases and 15-hydroxyprostaglandin dehydrogenase. Endocrinology (2004).
58. Kawamata, H. et al. Identification of genes differentially expressed in a newly isolated human metastasizing esophageal cancer cell line, T.Tn-AT1, by cDNA microarray. Cancer Sci 94, 699-706 (2003).
59. Laird, P. W. The power and the promise of DNA methylation markers. Nat Rev Cancer 3, 253-66 (2003).
60. Hermeking, H., Lengauer, C., Polyak, K., He, T. C., Zhang, L., Thiagalingam, S., Kinzler, K. W. & Vogelstein, B. (1997). Mol Cell, 1, 3-11.
61. Laird, P. W. (2003). Nat Rev Cancer, 3, 253-66.
62. Umbricht, C. B., Evron, E., Gabrielson, E., Ferguson, A., Marks, J. & Sukumar, S. (2001). Oncogene, 20, 3348-53.
63. Bismar, T. A., Humphrey, P. A., Grignon, D. J., and Wang, H. L. Expression of beta-catenin in prostatic adenocarcinomas: a comparison with colorectal adenocarcinomas. Am J Clin Pathol, 121: 557-563, 2004. 35.
64. Chesire, D. R., Ewing, C. M., Sauvageot, J., Bova, G. S., and Isaacs, W. B. Detection and analysis of beta-catenin mutations in prostate cancer. Prostate, 45: 323-334, 2000. 36.
65. de la Taille, A., Rubin, M. A., Chen, M. W., Vacherot, F., de Medina, S. G., Burchardt, M., Buttyan, R., and Chopin, D. Beta-catenin-related anomalies in apoptosis-resistant and hormone-refractory prostate cancer cells. Clin Cancer Res, 9: 1801-1807, 2003.
66. Civenni, G., Holbro, T., and Hynes, N. E. Wnt1 and Wnt5a induce cyclin D1 expression through ErbB1 transactivation in HC11 mammary epithelial cells. EMBO Rep, 4: 166-171, 2003.

1. A method for diagnosing prostate carcinoma comprising determining of the methylation degree in the genomic locus of a gene selected from the group consisting of 14-3-3σ, DDB2, GPX3, GSTM1, SFRP1, DKK3, p57/KIP2, COX-21PTGS2, HPGD and combinations thereof in a sample, wherein a hypermethylation is indicative for prostate cancer.

2. The method of claim 1 comprising determining the methylation degree of a CpG sequence.

3. The method of claim 1 comprising determining the methylation degree in the promoter region of said genes.

4. The method of claim 1 wherein the sample is a prostate tissue section or a body fluid.

5. The method of claim 1 wherein the sample is derived from a human patient.

6. The method of claim 1 wherein the determination comprises bisulfite sequencing.

7. The method of claim 1 wherein the determination comprises methylation-specific nucleic acid amplification, particularly methylation specific PCR (MSP).

8. The method of claim 1 wherein the determination comprises microarray analysis.

9. The method of claim 1 comprising determining of the methylation degree in the genomic locus of a first gene selected from the group consisting of 14-3-3σ, SFRP1, COX-2/PTGS2, GSTM1 and combinations thereof and in the genomic locus of a second gene selected from the group consisting of GPX3, DKK3 and combinations thereof.

10. A kit for diagnosing prostate carcinoma comprising reagents for determining of the methylation degree in the genomic locus of a gene selected from the group consisting of 14-3-3, DDB2, GPX3, GSTM1, SFRP1, DKK3, p57/KIP2, COX-2/PTGS2, HPGD and combinations thereof.

11. The kit of claim 10 wherein the reagents comprise (i) nucleic acid amplification primers and/or (ii) hybridization probes.

12. The kit of claim 10 for use in a method of diagnosing prostate carcinoma comprising determining of the methylation degree in the genomic locus of a gene selected from the group consisting of 14-3-3σ, DDB2, GPX3 GSTM1 SFRP1 DKK3, p57/KIP2, COX-21PTGS2, HPGD and combinations thereof in a sample, wherein a hypermethylation is indicative for prostate cancer.

13. A method for treating prostate carcinoma comprising modulating, e.g. decreasing the methylation degree in the genomic locus of a gene selected from the group consisting of 14-3-3s, DDB2, GPX3, GSTM1, SFRP1, DKK3, p57/KIP2, COX-2/PTGS2, HPGD and combinations thereof in a subject suffering from hypermethylation-associated prostate cancer.

14. The method of claim 13, wherein said subject suffering from hypermethylation associated prostate cancer is a human patient.

15. The method of claim 13 comprising administration of at least one demethylating agent in an amount which is sufficient to provide an at least partial demethylation of said genes.

16. The use of at least one demethylating agent for the preparation of a pharmaceutical composition for treating prostate carcinoma in a subject in need thereof by decreasing the methylation degree in the genomic locus of a gene selected from the group consisting of 14-3-3s, DDB2, GPX3, GSTM1, SFRP1, DKK3, p57/KIP2, COX-2/PTGS2, HPGD and combinations thereof.

17. The use of claim 16, wherein said subject in need thereof is a mammal, particularly a human patient.
